(19) United States
(12) Patent Application Publication
(10) Pub. No.: US 2025/0006380 A1
(43) Pub. Date: Jan. 2, 2025

(54) RISK DETECTION WITH ACTIONABLE GUIDANCE FOR NEURODEVELOPMENTAL DISORDERS

(71) Applicant: Duke University, Durham, NC (US)

(72) Inventors: Geraldine Dawson, Durham, NC (US); Guillermo Sapiro, Durham, NC (US)

(73) Assignee: Duke University, Durham, NC (US)

(21) Appl. No.: 18/759,522

(22) Filed: Jun. 28, 2024

Related U.S. Application Data
(60) Provisional application No. 63/523,761, filed on Jun. 28, 2023.
(60) Provisional application No. 63/523,764, filed on Jun. 28, 2023.

(51) Int. Cl. G16H 50/30; G16H 50/20

(52) U.S. Cl. CPC G16H 50/30; G16H 50/20

(57) ABSTRACT

The present disclosure describes methods and systems for risk detection and intervention for neurodevelopmental disorders. The method includes assessment of risk level, guidance and treatment recommendations and strategies, and longitudinal monitoring of patients with neurodevelopmental disorders. The assessments and monitoring can be integrated into the patient's health care program and electronic health record (EHR).

### CROSS REFERENCE TO RELATED APPLICATIONS

This application claims benefit of and priority to U.S. Provisional Patent Application Ser. No. 63/523,761, filed Jun. 28, 2023, the contents of which is herein incorporated by reference in its entirety; and claims benefit of and priority to U.S. Provisional Patent Application Ser. No. 63/523,764, filed Jun. 28, 2023, the contents of which is herein incorporated by reference in its entirety.

### GOVERNMENT INTEREST

This invention was made with government support under grant nos. HD093074, MH121329, and MH120093 awarded by the National Institutes of Health (NIH). The government has certain rights in the invention.

### TECHNICAL FIELD

The subject matter disclosed herein relates generally to systems and methods for detecting risk of neurodevelopmental disorders. More particularly, the subject matter disclosed herein relates to early identification and monitoring of children with potential Autism spectrum disorder.

### BACKGROUND

Autism spectrum disorder (ASD) is a neurodevelopmental disorder characterized by impairments in social communication and the presence of repetitive behaviors and restricted interests. With current prevalence estimates as high at 1 in 36 children, early identification and treatment of young children with ASD represents a significant public health and clinical care imperative and challenge. There is strong evidence that young children with ASD receiving early behavioral intervention services demonstrate substantial gains in functioning, and the American Academy of Pediatrics recommends screening all children for ASD at 18 and 24 months.

However, studies have exposed some inefficiencies in the current screening practices, which are comprised of parent questionnaires and parent interviews. Primary care givers may fail to utilize critical follow up questions, resulting in an excessive number of false positives that can cause undue anxiety to parents while also increasing wait times for those children at highest risk for ASD to receive specialized evaluation by an autism expert. Alternately, there is also some evidence of inflated false negatives among lower maternal socioeconomic status (SES), girls, and racial/ethnic minority populations, leading to poorer developmental outcomes of these autistic children.

Thus, there is an ongoing need for improved, objective, and scalable methods and systems for early identification and monitoring of children with potential ASD.

### SUMMARY

In accordance with this disclosure, systems and methods for detecting risk of neurodevelopmental disorders are provided. The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a method of detecting risk of neurodevelopmental disorders, comprising, consisting of, or consisting essentially of collecting patient information; deriving a risk level from the patient information; and providing next-step guidance corresponding to the risk level, where collecting the patient information comprises extracting information from one or more outputs, including but not limited to an electronically-administered screening survey; electronic health records information; direct observation of the patient via an application delivered on a computer, tablet, or smartphone; a phenotypic algorithm (such as a transformer-based neural network pre-trained on biomedical text); predictive markers, genetic testing; and any combination thereof.

In some embodiments, extracting information from direct observation of the patient includes observing the patient via the application and quantifying the patient's behavior using computer vision analysis and transmitting the patient information from the computer, tablet, or smartphone to a central healthcare data system.

In some embodiments, extracting information from a phenotypic algorithm includes collecting data from computer vision analysis of any of a variety of activities, including but not limited to attention, response to name, facial expression and dynamics, postural control, head movements, blink rate, and fine motor skills, social gaze, and any combination thereof.

In some embodiments, deriving the risk level includes deriving individual risk assessments from each of the one or more outputs and aggregating the individual risk assessments to determine the risk level. In some embodiments, for example, aggregating the individual risk assessments includes assigning weights to the individual risk assessments based on outputs from a machine learning algorithm. Individual risk assessments can include survey information, direct assessment of behavior via computer vision analysis, electronic health record information, and genetic testing.

In some embodiments, providing next-step guidance includes interfacing with a central healthcare data system to deliver recommendations for referrals and/or treatment.

In some embodiments, providing next-step guidance includes integrating strategies and/or monitoring to the caregiver and/or patient within a routine schedule of care.

Another aspect of the present disclosure provides a system for detecting and/or monitoring risk of neurodevelopmental disorders, comprising a computing system configured to perform the disclosed methods.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE FIGURES

The patent or application file contains at least one drawing executed in color. Copies of this patent or patent application publication with color drawings will be provided by the United States Patent and Trademark Office upon request and payment of the necessary fee.

FIG. 1. S2K-PT delivered in Duke Primary Care exam room.

FIG. 2. Frame-by-frame time-series for mean percent neutral expressions in autistic (red) and NT (blue) children. Carpenter et al., 2021.

FIG. 3. Time series of mean head movement rate during one movie for autistic (red) vs. NT (blue) children. Dawson et al., 2018.

FIG. 4. Percent right scores reflecting attentional preference for the toys versus the person for NT (blue) and autistic toddlers (orange) during a socially salient interval of the movie and the entire movie. Chang et al., 2021.

FIG. 5. Gaze-speech correlations for autistic, NT, and DDLD toddlers. Chang et al., 2021.

FIG. 6. Sensitivity and specificity for autism detection based on social attention features.

FIG. 7. Distributions of social attention gaze features for 36-96 month old autistic (orange) and NT (blue) children.

FIG. 8. S2K-TP administration.

FIG. 9. S2K-TP movies involve ethnically/racially diverse actors and are offered in English and Spanish.

FIG. 10 is a diagram illustrating an example computing platform for detecting and/or monitoring risk of neurodevelopmental disorders.

FIG. 11 is a diagram illustrating an example process for detecting and/or monitoring risk of neurodevelopmental disorders.

### DETAILED DESCRIPTION

The present subject matter provides systems and methods for detecting risk of neurodevelopmental disorders. For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical or caregiver intervention made in response to a disease, disorder or physiological condition manifested by a patient or to which a patient may be susceptible. The aim of treatment includes the alleviation or prevention of maladaptive symptoms, slowing or stopping the progression or worsening of a disease, disorder, or condition and/or the remission of the disease, disorder or condition and promotion of adaptive behavior and well-being.

As used herein, the term “subject” and “patient” are used interchangeably herein. In some embodiments, the subject comprises a human who is undergoing monitoring and/or treatment using systems and methods as prescribed herein. The term “effective amount” or “therapeutically effective amount” refers to an amount sufficient to effect beneficial or desirable biological and/or clinical results.

Unless otherwise defined, all technical terms used herein have the same meaning as commonly understood by one of ordinary skill in the art to which this disclosure belongs.

It is initially noted that, for simplicity, the terms “screening for ASD”, “detecting ASD”, etc. are used to generally refer to assessing behavioral patterns and other factors that are indicators of ASD, and which may or may not lead to a final diagnosis of ASD. In other words, the systems and methods described herein are designed to accurately identify individuals who have, or are at risk of developing, ASD. It is also envisioned that, although the description is directed toward the indication of ASD in particular, the systems and methods disclosed herein can potentially be applied to other neurodevelopmental disorders, such as attention deficit hyperactivity disorder (ADHD), receptive and/or expression language disorder or delay, intellectual disability, and/or developmental delay.

One common tool for screening children for ASD is a care giver survey, such as the Modified Checklist for Autism in Toddlers, Revised with Follow-Up (M-CHAT-R/F). Studies have shown that the use of an electronic M-CHAT-R/F when compared with a paper and pencil version ensures that all children scoring in the moderate likelihood range receive the recommended follow-up questions. Thus, the present disclosure builds on this premise by combining a digital survey (e.g., the M-CHAT-R/F or other digital survey), which asks the patient or the patient's caregiver about the presence and severity of autism symptoms, with novel algorithms and other elements that incorporate a number of screening factors to provide an accurate indication of potential ASD. The disclosed systems and methods additionally provide intervention in the form of targeted guidance and strategies based on an assessed risk level.

One aspect of the present disclosure provides a method of detecting risk of neurodevelopmental disorders. The method comprises collecting patient risk information from several sources and using a novel algorithm to assess a risk level and provide next-step guidance.

The information used to determine level of patient risk for a neurodevelopmental disorder based on multiple sources of risk information. These sources include one or more of the sources disclosed hereinbelow.

A first source of information is automated scoring of electronically-administered screening, in which a survey queries caregiver's and/or patient's knowledge of behavioral and medical risk factors. The screening survey can be, for example, the M-CHAT-R/F. The survey results are automatically calculated by the device or an associated peripheral program. This source of information can be comparable to existing screening systems.

A second source of information is based on additional electronic health record data that is typically collected from a clinical visit, including such as medical diagnoses and medical treatment information and provider notes.

A third source of information is an algorithm that uses direct observation of patient behavioral risk-related symptoms derived from a digital phenotyping application. The algorithm is based on active closed-loop sensing, where the patient is shown brief, developmentally-appropriate, dynamic stimuli on a computer, smart tablet, or smartphone, while the sensors in the same device capture information for automatic, objective quantification of several behavioral risk markers. Some examples of behavioral risk markers include but are not limited to patterns of attention, gaze, orienting, facial movements and expressions, oral kinetics, posture, touch, blink rate, and motor responses. The application can also include interactive games, which can detect patterns of motor behavior. The application can be deployed in a clinical setting, or it can be used remotely by parents and/or home caregivers independently, with results from the activities being stored for later retrieval or transmitted for analysis at a central location.

A fourth source is of information genetic testing. This can include, for example, chromosomal microarray analysis, exome and/or whole genome sequencing, and tests designed to detect point mutations. These can be carried out on blood, saliva, and/or buccal swab samples.

Once data from the various information sources has been assembled, a composite neurodevelopmental risk assessment is calculated. Broadly, if each source of information is considered as a risk question having a yes/no response, the overall risk can be calculated as the fraction of positive responses. This overall risk can be calculated in a suitable fashion and is not limited to a particular algorithm. For example, the composite risk can be a simple summation of positive responses, or the information sources can each be assigned a weight. In a non-limiting example, genetic information may be given a lower weight than behavioral testing. In another example, certain score combinations can be multiplicative, and/or machine learning can be used to compare subject scores to datasets. Such an application of machine learning can account for differences in the number of data sources from which result can be derived, identify correlations between different tests, and/or apply appropriate weights to different inputs. Further, the overall score can be assigned a general score (e.g., high, medium, low), or it can be separated into multiple categories (e.g., based on types of behavioral challenges).

The next step of the method is to provide automated clinical decision support based on the results of the risk assessment. In some embodiments, the clinical decision support can include guidance that is tailored to the specific results of the assessment. This can be, for example, a recommendation for a next level of testing or a specific treatment. The automated nature of the guidance advantageously provides a standardized referral and guidance framework that reduces the need for clinical judgement. The guidance can be directed to the provider, caregiver, and/or patient regarding referrals and linkage to services that, in the future, can be integrated into pediatric primary care. In addition, the next-step guidance can be provided in a manner that integrates with the existing healthcare pipeline to provide actionable guidance at an appropriate time without disrupting the regular schedule of care. In this regard, the present systems and methods can be designed to interface with an existing healthcare data system to deliver recommendations for referrals and/or treatment at well-child visits, at previously scheduled appointments, and/or otherwise within a routine schedule of care.

In some embodiments, the method optionally comprises providing strategies and/or ongoing monitoring for patient outcomes, either symptom worsening or symptom improvement. The strategies and monitoring can be provided in any suitable method, such as on a computer, tablet, or smartphone. Some non-limiting examples of guidance include prompts to seek appropriate follow up evaluation and fillable forms that indicate any useful evaluation information such as type, evaluator, time, and location of an evaluation, as well as evaluation results. Additional guidance can include caregiver and/or patient coaching strategies that can be used by a caregiver and/or patient to promote the patient's learning, adaptive behavior, social interaction, communication skills, etc.

Monitoring can include the monitoring and recording patient symptoms, as well as detecting changes in symptoms. Changes may be detected, for example, by routine re-evaluation of the patient using the algorithms described herein or by conventional approaches. In an example, the above-mentioned phenotyping algorithm can be administered at prescribed intervals. The results can be recorded and analyzed for significant changes or trends.

Another aspect of the present disclosure provides a system for identifying and monitoring neurodevelopmental disorders of a subject. The system comprises any suitable elements necessary for configured to perform to the disclosed method. These components will be understood by a person of skill in the art and are not described in detail herein. This can include, but is not limited to a computer having a processor and a memory configured to execute the algorithms and user interface device(s) such as a tablet, smartphone, camera, video display, etc.

The disclosed method and system advantageously provide automated integration of information from surveys and/or direct observation of patient symptoms via the digital phenotyping app about patient's longitudinal course of symptoms into the EHR.

Another aspect of the present disclosure provides all that is described and illustrated herein.

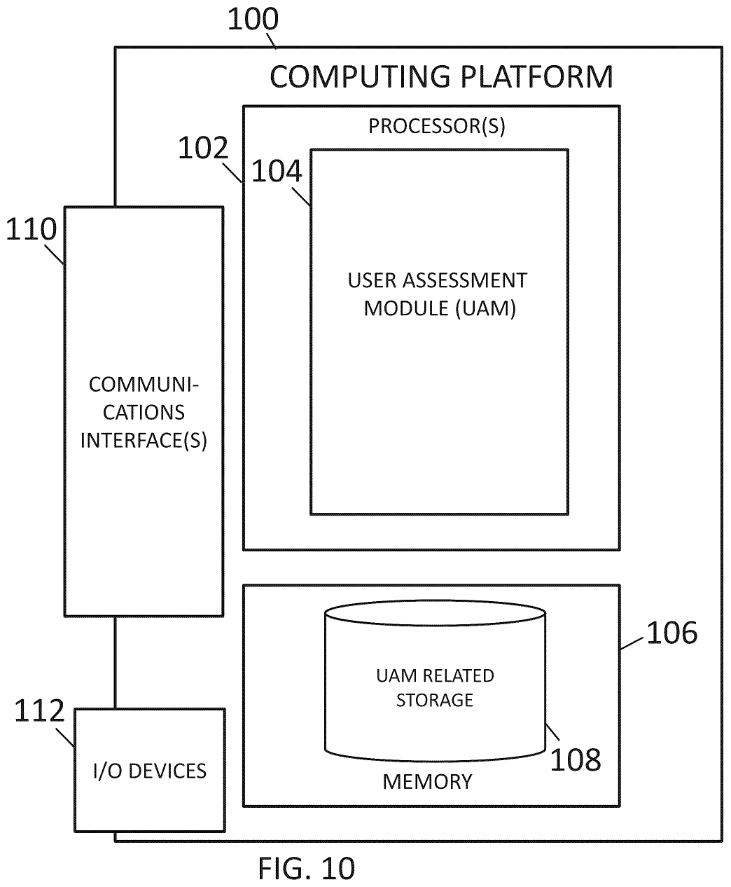

In some embodiments, referring to FIG. 10, computer platform 100 or a related module may include functionality for providing next-step guidance corresponding to the risk level using patient information (e.g., information from one or more outputs selected from the group consisting of an electronically-administered screening survey; electronic health records information; direct observation of the patient via an application delivered on a computer, tablet, or smartphone; a phenotypic algorithm; predictive markers; genetic testing; and any combination thereof, which can be obtained from or derived from user interaction with one or more apps executing on computer platform 100 or another device). In such embodiments, the next step guidance may include a prediction value related to a diagnosis (e.g., indicating a risk that a user has a neurodevelopmental/psychiatric disorder) along with a prediction confidence value indicating a confidence or likelihood that the prediction value is accurate and a quality value indicating whether an assessment should be readministered (e.g., because the metrics (e.g., app-derived features) are poor quality or insufficient for an accurate assessment).

Computing platform 100 may include processor(s) 102. Processor(s) 102 may represent any suitable entity or entities (e.g., one or more hardware-based processor) for processing information and executing instructions or operations. Each of processor(s) 102 may be any type of processor, such as a central processor unit (CPU), a microprocessor, a multi-core processor, and the like. Computing platform 100 may further include a memory 106 for storing information and instructions to be executed by processor(s) 102.

In some embodiments, memory 106 can comprise one or more of random access memory (RAM), read only memory (ROM), static storage such as a magnetic or optical disk, or any other type of machine or non-transitory computer-readable medium. Computing platform 100 may further include one or more communications interface(s) 110, such as a network interface card or a communications device, configured to provide communications access to various entities (e.g., other computing platforms). In some embodiments, one or more communications interface(s) 110 may include a user interface configured for allowing a user (e.g., a diagnostic subject for assessment or an assessment operator) to interact with computing platform 100 or related entities. For example, a user interface may include a graphical user interface (GUI) for providing a questionnaire to user and/or for receiving input from the user and/or for displaying region-based stimuli to a user. In some embodiments, memory 106 may be utilized to store a user risk assessment module (UAM) 104, or software therein, and a UAM related storage 108.

UAM 104 may be any suitable entity (e.g., software executing on one or more processors) for performing one or more aspects associated with user assessment. In some embodiments, UAM 104 may be configured for detection of a risk of a neurodevelopmental or psychiatric disorder using phenotyping. For example, UAM 104 may be configured for obtaining and using patient information as discussed herein.

Memory 106 may be any suitable entity or entities (e.g., non-transitory computer readable media) for storing various information. Memory 106 may include UAM related storage 108. UAM related storage 108 may be any suitable entity (e.g., a database embodied or stored in computer readable media) storing user data, stimuli (e.g., digital content, games, videos, or video segments), recorded or captured responses, and/or predetermined information. For example, UAM related storage 108 may include machine learning algorithms, algorithms for statistical analysis, SHAP analysis, and/or report generation logic. UAM related storage 108 may also include user data, such as age, name, knowledge, skills, sex, and/or medical history. UAM related storage 108 may also include predetermined information, including information gathered by clinical studies, patient and/or caregiver surveys, and/or doctor assessments.

In some embodiments, predetermined information may include information for analyzing responses; information for determining based responses; information for determining assessment thresholds; coping strategies; recommendations (e.g., for a caregiver or a child); treatment and/or related therapies, information for generating or selecting games, videos, video segments, digital content, or related stimuli usable for a user assessment; and/or other information.

In some embodiments, UAM related storage 108 or another entity may maintain associations between relevant health information and a given user or a given population (e.g., users with similar characteristics and/or within a similar geographical location). For example, users associated with different conditions and/or age groups may be associated with different recommendations, base responses, and/or assessment thresholds for indicating whether user responses are indicative of neurodevelopmental/psychiatric disorders.

In some embodiments, UAM related storage 108 may be accessible by UAM 104 and/or other modules of computing platform 100 and may be located externally to or integrated with UAM 104 and/or computing platform 100. For example, UAM related storage 108 may be stored at a server located remotely from a mobile device containing UAM 104 but still accessible by UAM 104. In another example, UAM related storage 108 may be distributed or separated across multiple nodes.

It will be appreciated that the above described modules or entities are for illustrative purposes and that features or portions of features described herein may be performed by different and/or additional modules, components, or nodes. For example, aspects of user assessment described herein may be performed by UAM 104, computing platform 100, and/or other modules or nodes.

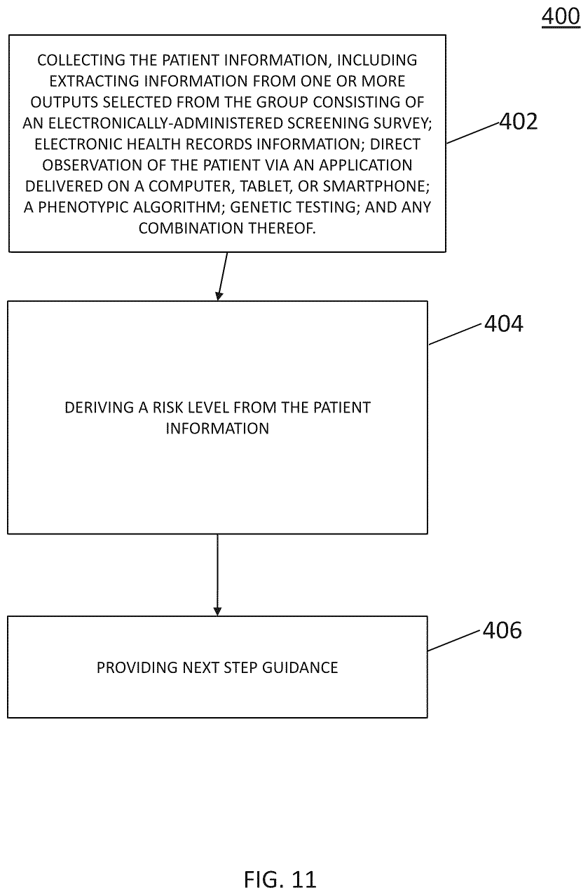

FIG. 11 is a diagram illustrating an example process 400 for detecting risk of neurodevelopmental disorders. In some embodiments, process 400 described herein, or portions thereof, may be performed at or by computing platform 100, UAM 104, and/or another module or node. For example, computing platform 100 may be a mobile device, a computer, or other equipment (e.g., a computerized chair or room) and UAM 104 may include or provide an application running or executing on computing platform 100. In some embodiments, process 400 may include steps 402-406.

In step 402, patient information may be obtained. In some embodiments, collecting the patient information comprises extracting information from one or more outputs selected from the group consisting of an electronically-administered screening survey; electronic health records information; direct observation of the patient via an application delivered on a computer, tablet, or smartphone; a phenotypic algorithm; genetic testing; and any combination thereof. In some embodiments, the information comprises predictive markers, which in some embodiments comprise patterns of early medical conditions. In some embodiments, a machine learning algorithm is used to identify the predictive markers. In some embodiments, the machine learning algorithm comprises a transformer-based neural network pre-trained on biomedical text. In some embodiments, the extracting information from direct observation of the patient comprises: observing the patient via the application using computer vision analysis; and transmitting the patient information from the computer, tablet, or smartphone to a central healthcare data system. In some embodiments, the extracting information from a phenotypic algorithm comprises collecting data from computer vision analysis of activities selected from the group consisting of response to name, facial expression and dynamics, postural control and fine motor skills, social attention, and any combination thereof.

In step 404, the method comprises deriving a risk level from the patient information. In some embodiments, deriving the risk level comprises: deriving individual risk assessments from each of the one or more outputs; and aggregating the individual risk assessments to determine the risk level. In some embodiments, the aggregating the individual risk assessments comprises assigning weights to the individual risk assessments based on outputs from a machine learning algorithm.

In step 406, next step guidance is provided. In some embodiments, providing next-step guidance comprises interfacing with a central healthcare data system to deliver recommendations for referrals and/or treatment. In some embodiments, providing next-step guidance comprises integrating strategies and/or monitoring to the caregiver and/or patient within a routine schedule of care. For example, a next step guidance may be generated using UAM 104 and provided to a user (e.g., a subject, a parent, or a medical provider), e.g., via a display device and/or communications interface(s) 110 (e.g., a GUI). In another example, a user assessment report may be generated and stored in memory 106 or UAM related storage 108.

In some embodiments, computing a prediction confidence value may include performing a model interpretability analysis (e.g., a SHAP analysis, a LIME analysis, a permutation importance analysis, a feature importance analysis, etc.) involving metrics and a machine learning based model. For example, using SHAP or LIME analysis, UAM 104 or another entity may explain or interpret how various app variables (e.g., app-derived metrics) affect the model's behavior or output. In this example, using normalized SHAP interaction values, UAM 104 or another entity may identify the relative importance of each potential app variable to the model's output, e.g., at an overall level or population level. Continuing with this example, UAM 104 or another entity may also use normalized SHAP interaction values for the actual app variables obtained for a particular user (e.g., the actual app variables may be a subset of the potential app variables that can be obtained or derived) to determine how those particular app variables affected the user's particular prediction value generated by the model.

In some embodiments, performing a model interpretability analysis may include generating normalized SHAP interaction values associated with app-derived metrics and using the normalized interaction values in generating a user-specific prediction profile indicating how the user's metrics affected the user's diagnosis or prediction value. For example, SHAP value analysis may provide information about the relative contribution of each of the potential app-derived metrics to the prediction output (e.g., ASD or neurotypical) of a model (e.g., at a population level) and may also provide information usable when generating a user's unique profile indicating what specific metrics (e.g., the patient information) and to what extent these metrics contributed to the user's prediction value.

In some embodiments, a machine learning based model may include an XGBoost algorithm. For example, a trained XGBoost model may comprise or utilize multiple decision trees (e.g., 1,000 trees) that are trained using 5-fold cross-validation where the data is shuffled to compute individual intermediary binary predictions.

In some embodiments, a neurodevelopmental/psychiatric disorder may an ASD, an ADHD, or an anxiety disorder diagnosis.

In some embodiments, computing platform 100 may include a mobile device, a smartphone, a tablet computer, a laptop computer, a computer, a user assessment device, or a medical device.

It will be appreciated that process 400 is for illustrative purposes and that different and/or additional actions may be used. It will also be appreciated that various actions described herein may occur in a different order or sequence.

It should be noted that computing platform 100, UAM 104, and/or functionality described herein may constitute a special purpose computing device. Further, computing platform 100, UAM 104, and/or functionality described herein can improve the technological field of for detecting and/or monitoring risk of neurodevelopmental disorders by providing mechanisms for early for detecting and/or monitoring risk of neurodevelopmental disorders. Moreover, such mechanisms can alleviate various barriers, including costs, equipment, and human expertise, associated with conventional (e.g., clinical) methods of detecting and/or monitoring risk of neurodevelopmental disorders.

It should also be noted that computing platform 100 that implements subject matter described herein may comprise a special purpose computing device usable for various aspects of for detecting and/or monitoring risk of neurodevelopmental disorders. Outputs can be used to prioritize assessment and therapy services in the context of long wait-lists for such services.

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

The disclosure of each of the following references is incorporated herein by reference in its entirety to the extent that it is not inconsistent herewith and to the extent that it supplements, explains, provides a background for, or teaches methods, techniques, and/or systems employed herein.

### REFERENCES

1. Chang Z, Di Martino J M, Aiello R, et al. Computational Methods to Measure Patterns of Gaze in Toddlers With Autism Spectrum Disorder. JAMA Pediatr 2021; 175(8): 827-36.
2. Krishnappa Babu P R, Di Martino M, Chang Z, et al. Exploring complexity of facial dynamics in autism spectrum disorder. IEEE Trans Affect Comput 2021.
3. Krishnappa Babu P R, Di Martino J M, Chang Z, et al. Complexity analysis of head movements in autistic toddlers. J Child Psychol Psychiatry 2023; 64(1): 156-66.
4. Perochon S, Di Martino M, Aiello R, et al. A scalable computational approach to assessing response to name in toddlers with autism. J Child Psychol Psychiatry 2021; 62(9): 1120-31.
5. Krishnappa Babu P R, Aikat V, Di Martino J M, et al. Blink rate and facial orientation reveal distinctive patterns of attentional engagement in autistic toddlers: a digital phenotyping approach. Scientific Reports 2023, 13(1): 7158.
6. Perochon S, Di Martino J M, Carpenter K L H, et al. A tablet-based game for the assessment of visual motor skills in autistic children. npg Digit Med 2023; 6(1): 17.
7. Perochon, S., Di Martino, J. M., Carpenter, K. L. H., Compton, S., Davis, N., Eichner, B., Espinosa, S., Franz, L., Krishnappa Babu, P. R., Sapiro, G., and Dawson, G. (2023). Early detection of autism using digital behavioral phenotyping. Nature Medicine. 2023 October; 29(10):2489-2497. PMID: 37783967.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

Additional details and example methods, mechanisms, techniques, and/or systems for early detection of autism or related aspects are further described in the Examples that follow.

### Example

The following EXAMPLE provide illustrative embodiments. In light of the present disclosure and the general level of skill in the art, those of skill will appreciate that the following EXAMPLE is intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLE, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example—A Digital Health Approach to Early Identification and Outcome Monitoring in Autism

This Example evaluates novel digital behavioral assessment tools based on computer vision analysis (CVA) and machine learning (ML) that can be implemented on widely available devices in real-world settings to improve the accuracy of autism screening and enable longitudinal monitoring of children's behavior and development. The standard of care for autism screening at 18-24 months of age is a caregiver questionnaire, the Modified Checklist for Autism in Toddlers-Revised with Follow Up (M-CHAT-R/F).¹ Although useful,² the M-CHAT-R/F has lower accuracy in real-world settings, such as primary care,³ and with caregivers from Black and Hispanic/Latino backgrounds, those with lower education, and with girls.³⁻⁶ These disparities are notable given documented delays in autism diagnosis for children of color and girls.^{7, 8} This Example provides an objective and feasible method for the direct observation and quantification of autism-related behaviors.⁹⁻¹³ We developed an application (app), SenseToKnow (S2K), administered on a smartphone or tablet, which displays brief, strategically designed movies while the child's behavioral responses are recorded via the frontal camera embedded in the device, and uses CVA and ML to quantify the child's behavior (digital phenotypes). Collaborating with primary care clinics, we conducted a proof-of-concept study for a Toddler-Preschool version of the app (S2K-TP) with a sample of 993 16-38-month-old children (40 diagnosed with autism) and demonstrated its ability to distinguish autistic and neurotypical (NT) children.¹³ This Example expands upon this work. First, it is determined whether S2K-TP administered at 16-30 months of age can effectively be delivered remotely by parents at home, without sacrificing accuracy of the app for autism detection. This would increase the tool's scalability and accessibility. Second, it is examined how race, ethnicity, sex, family income, and maternal education influence S2K-TP's accuracy, given that these factors affect performance of the M-CHAT-R/F. Third, it is evaluated whether S2K-TP can serve as an objective method for outcome monitoring by examining whether the S2K-TP digital phenotypes have convergent validity compared to standardized clinical measures. This allows us to determine whether S2K-TP is effective for assessing autism-related behaviors in preschool age children. Fourth, with a goal of expanding the types of behavioral measures that could be used for screening and monitoring, the feasibility of using CVA to measure parent-child interaction from videos recorded at home is explored. Finally, in collaboration with EXAMPLE 2, the perceived usability of automated clinical decision support (CDS) for primary care providers (PCPs) that integrates autism screening information with actionable guidance regarding referrals for diagnosis and services is designed and assessed.

This Example pertains to the following aims:

Aim 1—Assess the accuracy of a remotely delivered digital phenotyping app (S2K-TP) for autism detection in a diverse population of 16-30-month-old participants recruited through Duke Primary Care clinics. The sensitivity, specificity, negative/positive predictive values and test-retest reliability of S2K-TP delivered by parents at home for autism detection and the influences of race, ethnicity, sex, family income, and maternal education on S2K-TP's accuracy are assessed and examined.

Aim 2—The convergent validity of S2K-TP by evaluating the concurrent and longitudinal relationships between S2K-TP digital autism-related phenotypes (response to name, facial expressions and dynamics, postural control, fine motor skills, social attention and standardized clinical measures of autism-related behaviors with autistic children at 16-30, 36, and 48 months is examined.

Aim 3—The feasibility of using CVA to measure patterns of parent-child interactions videotaped at home is explored. With 50 16-30-month-old autistic and 50 neurotypical (NT) children, the convergent validity of CVA measures is evaluated by testing the correlations between CVA measures of synchrony, proximity, and orientation, human coding of these constructs, and global ratings of parent-child joint engagement.

Aim 4—The perceived usability of CDS for autism screening is designed and assessed and a list of key priority factors for using autism screening CDS across a broad range of health care settings is assessed. Through engagement with a wide range of stakeholders, we will design and assess the perceived usability of a CDS prototype. We will iteratively design the CDS, using implementation science and human-centered methods to understand user contexts, obtaining measures of perceived usability from stakeholders. Through the design process, we will identify a set of key priority factors to consider when integrating a CDS for autism screening into primary care that is applicable to a broad range of health care settings.

This Example provides cutting-edge computational methods to develop innovative translational digital health tools that can address critical needs for improved autism screening, objective and sensitive behavioral outcome measures, and biomarkers for use in trials evaluating treatments for young autistic children.

### Research Strategy

In the U.S., 1 in 54 children is diagnosed with autism, and the annual economic cost of autism is estimated to be $268 billion with $461 billion forecasted by 2025.14 Timely access to accurate screening, referrals, and diagnosis is the critical first step to receiving early intervention, which results in better outcomes, quality of life, and cost-savings during a lifetime.¹⁴⁻¹⁷ Consistent with the priorities of the ACE RFA and the Interagency Autism Coordinating Committee (IACC) Strategic Plan, there remains an urgent need for new tools for detecting autism that can facilitate timely linkage to early intervention and support.18

The American Academy of Pediatrics (AAP) recommends screening for all toddlers in primary care.¹⁹ The most common method is a caregiver survey, the M-CHAT-R/F.1 Although useful, 2 mounting evidence suggests that the M-CHAT-R/F has significant limitations.^{3, 20-23} It has lower accuracy in primary care settings³ and with caregivers from Black and Hispanic/Latino backgrounds and lower education, and with girls.³⁻⁶ Compared to white parents, Black parents report fewer autism concerns.⁵ A substantial underuse of screening in primary care is due to difficulty interpreting caregivers' reports and the requirement to conduct a follow-up interview to increase the screening accuracy.¹ Even when near universal screening is achieved, a study of over 25,000 children found that the M-CHAT-R/F's sensitivity was 38.8% and its positive predictive value was 14.6%, with lower values for girls, children of color and lower-income households.3 While new screening questionnaires can help address these concerns,24-26 there remains a need for complementary tools that can directly measure children's behavior to address barriers related to literacy, language, and education inherent in questionnaires.⁹

Relatedly, the field has been hampered by the lack of objective and sensitive ways of assessing longitudinal changes in behavior.²⁷⁻³⁰ Because autism symptoms emerge over time, and some children lose previously-acquired skills, measuring a child's behavioral trajectory is important.³¹⁻³⁴ Tools are also needed to measure a child's response to treatment. Caregiver reports can be influenced by expectancy effects, and clinical ratings require extensive training and expert raters, making them less feasible for clinical trials.^{28, 35, 36}

How digital health approaches can address the current challenges in autism screening. In other fields of medicine and health, much progress has been made by using data-driven digital strategies which provide quantitative and objective measures in combination with subjective clinical assessments, such as caregiver report. Combining digital approaches with clinical measures, such as caregiver report, has been shown to provide a more accurate screening method.³⁷ The fields of psychiatry and psychology have yet to fully realize the potential of this model.^{9, 38} We have developed a digital assessment tool for autism-related behaviors that can be delivered on widely-available mobile devices by designing an application (app; S2K) comprising brief strategically designed movies shown on a smartphone or tablet while the child's behavior is recorded via the frontal camera embedded in the device, and using computer vision analysis (CVA) and machine learning (ML) to quantify behavioral responses. The Toddler-Preschool version of S2K (S2K-TP) also includes a bubble-popping game to assess motor skills and repetitive behaviors quantified by kinetic sensors.³⁹ We demonstrated that S2K-TP can elicit and quantify multiple known and novel autism-related phenotypes, including orienting to name,^{10, 40} facial expressions and dynamics,^{11, 41, 42} postural control,¹² fine motor skills,³⁹ and social attention,¹³ each of which distinguishes autistic and neurotypical (NT) toddlers. For example, one of the earliest autism symptoms is decreased attention to social stimuli, evident by 6 months of age.^{43, 44} Frazier et al. argued that adding eye-tracking to assess social attention to standard screening approaches could improve screening and decrease the lifetime costs of autism.⁴⁵ However, current eye-tracking methods require expensive equipment and trained personnel, limiting their use for universal screening and longitudinal monitoring, especially in low resource settings.^{46, 47} It was demonstrated for the first time that an app deployed on a smart phone or tablet can reliably measure gaze and detect patterns of social attention that distinguished autistic and NT toddlers, without the need for trained staff, calibration, or eye-tracking equipment.¹³

Independent evaluation with a larger sample is essential to (a) assess the feasibility and accuracy of S2K-TP when administered remotely at home, and (b) determine whether demographic factors influence the accuracy of S2K-TP. It is believed that S2K-TP, which quantifies direct behavior observations, is simple to administer and does not require literacy, knowledge of child development, or a follow-up interview, will identify toddlers who have a higher likelihood of autism and provide a valid method for monitoring children's behavior over time. It is also believed that S2K-TP, which offers finer resolution and dimensionality than caregiver report in quantifying behaviors, will reliably detect autism-related behaviors in girls, who have more subtle social features of autism and are more likely to be missed during screening.^{8, 48, 49}

Can computer vision analysis meaningfully capture variation in parent-child interaction? Assessments of caregiver-child interaction are part of a gold-standard autism diagnostic evaluation and a way of assessing outcomes of caregiver-delivered interventions.⁵⁰⁻⁵³ Such measures can complement caregiver reports and those derived from S2K-TP, which are elicited responses to stimuli. Caregiver-child interactions are usually assessed with clinical ratings, which lack granularity and sensitivity to change or require labor intensive coding.⁵⁴ This is not well-suited for large clinical trials. Digital assessment potentially offers a way to automatically capture meaningful aspects of caregiver-child interaction. From videos of diagnostic assessments, Kojovic et al. used 2D video-based pose estimation and trained a deep neural network on aspects of social interaction of children playing with an adult and achieved an accuracy of differentiation between autistic and NT children of 80.9%.⁵⁵ An exploratory aim is to evaluate the feasibility of using CVA to measure social interactions from videos of parent-child interaction recorded on a parent's device at home. A protocol for recording and coding of parent-child interaction via telehealth is employed and supports the potential value of CVA for measuring parent-child interaction. Automated CVA measures of parent-child interaction will be able to meaningfully quantify patterns of parent-child interaction and show adequate convergent validity compared to human coding and clinical ratings.

Designing a digital screening tool in the context of a clinical care pathway. Successful implementation of digital health tools involves combining the rigor of clinical and computational science with stakeholder engagement so that new tools can be effectively integrated in a clinical care workflow.³⁸

Digital health tools that have the greatest impact are easy to use, useful, and contextually appropriate. S2K-TP was developed using human-centered design (HCD) principles in real world settings (pediatrician exam rooms) that considered how S2K-TP could feasibly be integrated into the typical clinical workflow and health system.⁵⁶

S2K was designed in collaboration with pediatricians and caregivers, with iterative design and feedback regarding its perceived usability. This Example designs a clinical decision support (CDS) that combines autism screening information with actionable guidance for providers regarding appropriate referral for evaluation and services.^{57, 58} In a large study of pediatric practices, 69% of children with a positive autism screen were not referred for a diagnostic evaluation.⁵⁹ Hispanic and Black families, in particular, face difficulties receiving referrals for an autism evaluation.⁶⁰⁻⁶⁴ We examined the impact of providing CDS that included guidance for PCPs regarding appropriate referrals after M-CHAT-R/F screening and found a substantial increase in referral rate and a significant reduction in the mean age of diagnosis (see Preliminary data).^{65, 66}

Impact of the proposed research on a broader understanding of autism. This Example impacts the broader understanding of autism in several ways: (1) A remote, digital assessment could reach a more diverse population, reducing barriers related to language, literacy, and geography that exist when assessment only occurs in a provider's office. For example, screening could occur remotely and caregivers could be contacted proactively to encourage them to see a provider; (2) Given the viability of delivering early interventions via telehealth,⁶⁷⁻⁷⁰ a reliable tool for remote longitudinal monitoring would address a critical need for outcome measures for telehealth clinical trials;^{28, 71} (3) The ability to measure behavior remotely, including eye tracking, opens vast possibilities for gathering large data sets in real-world settings that can link digital phenotypes to genetic and other biosamples 72 and are amenable to big data analysis;^{73, 74} and (4) The higher resolution of CVA for measuring behavior allows for the discovery of novel phenotypes that are not detectable with the human eye, thereby providing new insights into the nature of autism.

As described in the ACE RFA and the IACC Strategic Plan,¹⁸ there remains a critical need to develop autism screening tools that are easy to use, objective, and designed to mitigate the well-established disparities in early detection and access to services. By developing digital health tools for autism screening and outcome monitoring, we aim to reduce barriers in access to screening related to feasibility, literacy, language, and geography. Because these tools are scalable, the implementation of such tools in health care institutions, homes, community settings, and clinical trials could have broad and substantial impact on the long-term outcomes and quality of life of autistic individuals and their families.

### Data for Aims 1 and 2

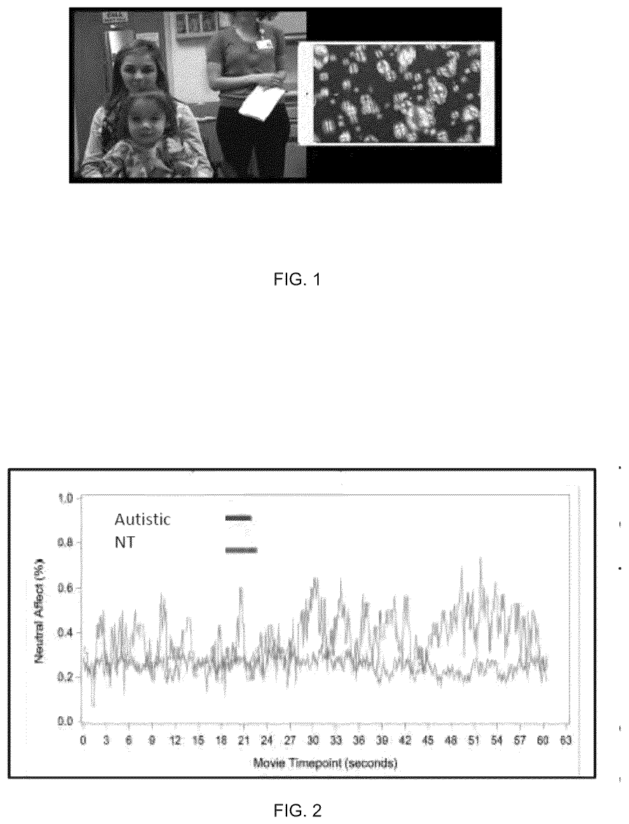

Proof-of-concept studies with 2K-TP at 16-36 months. We conducted an initial study (Study 1; beta version of S2K, iPad only) with 108 16-31-month-old toddlers (22 diagnosed with autism). In our current ACE, we conducted a second study (Study 2; current version—S2K-PT, iPad, iPhone) with 993 16-38-month-old toddlers (40 diagnosed with autism). Both were conducted with ethnically/racially diverse populations during an 18-24-month well-child visit in Duke pediatric primary care clinics (see FIG. 1).

Child engagement during app administration. Percentages of children who attended the majority of time were 95% and 87% for the iPhone and 95% and 93% for the iPad, for NT and autistic groups, respectively. Response to name. Not responding to name is a well-documented early indicator of autism.83-85 While the child watched brief movies, at pre-specified time points, the child was called by their name. Using CVA to measure the child's head turn, in Study 1, we found that NT toddlers oriented more times out of the 3 trials (B=1.89, P<0.05). Only 1 autistic toddler oriented twice, and none oriented three times. For those who responded, mean latency to orient for NT toddlers was 1.21 sec (SD=0.85), compared to 2.19 sec (SD=1.43) for autistic toddlers (P<0.05),40 differences not readily detectable by the human eye. Human coding of orienting demonstrated high reliability with computer coding (Intraclass correlation coefficient [ICC]=0.89). Findings were replicated in Study 2; 10 NT toddlers were more likely to respond to their name >2 times (Ps<0.005, 0.05 and 0.005 for the iPad, iPhone, and combined samples, respectively). The proportion of autistic toddlers who oriented <2 times was 0.78 vs. 0.43 for TD toddlers (F=6.5, P=0.001). As in Study 1, autistic children who oriented exhibited a longer latency compared to NT children (P<0.0001). The area under the curve (AUC) for tree-based classifiers that used both frequency and latency features showed differentiation between the groups (AUC=0.83 for iPad and 0.78 for iPhone). The two devices showed similar levels of feasibility (engagement by toddlers) and accuracy. Facial expression and dynamics. Autism is associated with differences in facial expression, and reduced affective expression is part of the diagnostic criteria for autism.⁸⁶⁻⁸⁸ In Study 1, CVA of children's facial responses revealed that autistic toddlers more frequently exhibited a neutral facial expression (P<0.005) than NT toddlers (see FIG. 2).¹¹

Human coding of neutral, positive, and negative affect showed strong agreement with CVA, with concordance of 0.89, 0.90, and 0.89, respectively.89 In Study 2, this finding was replicated (P<0.05). Multiscale entropy analysis captured distinctive landmarks dynamics in autistic children, characterized by higher complexity and lower predictability compared to NT children. During movies with non-social content (P<0.01/0.05), effect sizes were in the 0.32-0.40 range, compared to the social movies (P<0.0001), for which effect sizes were large to very large (0.51-0.82).⁴²

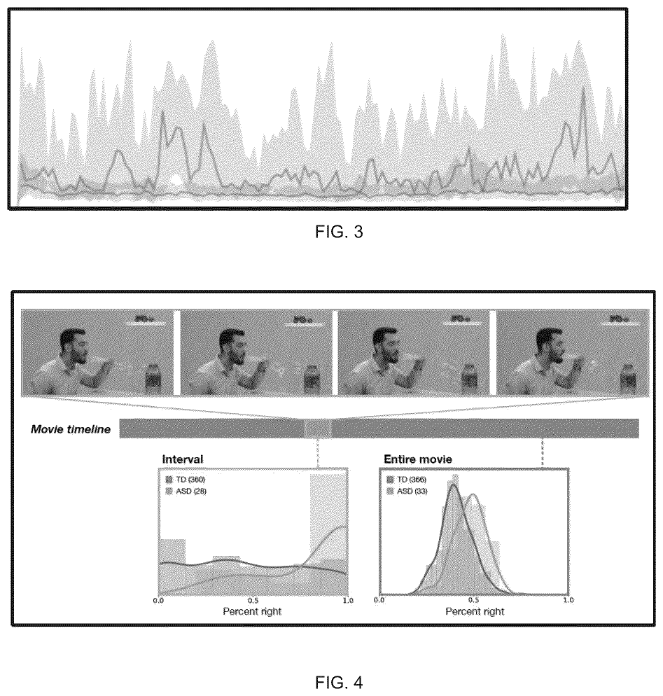

Postural control and fine motor skills. Delays in motor abilities are an early feature of autism⁹⁰ and predict later language acquisition.⁹¹ Reduced postural control (head movement) has been documented among autistic individuals and may reflect attentional demands when viewing complex, audiovisual stimuli.⁹²⁻⁹⁶ In Study 1, we used CVA to assess midline head postural control, as reflected in the rate of spontaneous head movements12. Autistic toddlers exhibited significantly higher postural sway compared to NT toddlers, accentuated during movies with social content (see FIG. 3). This finding was replicated in Study 2; Autistic toddlers showed higher head movement rate (P<0.001) and acceleration (P<0.0001).

S2K-TP also measures motor skills via an embedded bubble popping game. The tendency to return to the same bubble repeatedly assesses repetitive behavior. Touch is measured via kinetic sensors in the device. Autistic toddlers demonstrated lower fine motor control and accuracy than NT toddlers, reflected in the median error (distance between the center of the popped bubble and child's touch; P<0.005, r (effect size)=0.42,) and in bubble popping rate (ratio of the number of bubbles popped over the total number of touches; P<0.05, r=0.31).39

Social attention. S2K-TP includes movies designed to assess a child's preference for attending to social versus non-social stimuli (social attention), which depict a person on one side and a toy (played with by the person) on the opposite side of the screen (see FIG. 4). A toy on a shelf is in the top corner on the ‘toy’ side of the screen. In Study 2, a man was shown blowing bubbles (‘Blowing bubbles’) with a wand (iPad) or a bubble gun (iPhone). Two movies showed a woman spinning a top (iPad: ‘Spinning top’) or a pinwheel (iPhone: ‘Spinning pinwheel’). Similar to a probe used in the Autism Diagnostic Observation Schedule (ADOS)⁹⁷, at one point, the person pauses expectantly before blowing bubbles or spinning the toy. Dependent variable was the percent of frames looking to the right side of the screen (‘percent right’), reflecting the amount of time spent looking at the person or the toys, depending on which side of the screen the person and toys were displayed (counterbalanced; note that depending on the balanced side of the social and non-social stimuli, the percent right is expected to be higher or smaller for autistic participants, which is accounted for during the analysis). Another movie assessed attention to speech and depicted two women facing each other who talked about a trip to the park. A clock was in the upper right-hand corner. We measured the degree to which gaze followed the person who was speaking (gaze-speech time correlation). Analyses of percent right scores revealed differences between the NT and autistic groups, characterized by reduced gaze toward the person by autistic toddlers (‘Spinning pinwheel’ (iPhone): P<0.0001, r=0.51; ‘Blowing bubbles’ (iPhone): P<0.007, r=0.35; ‘Spinning top’ (iPad): P<0.01, r=0.33; ‘Blowing bubbles’ (iPad): P<0.0001, r=0.47). Examining gaze patterns when the actor paused expectantly, the group effect size increased from medium to large (0.47 to 0.59; See FIG. 4). Analysis of gaze during the movie involving two people having a conversation revealed differences between the autistic and NT groups for gaze-speech time correlations (P<0.0001, r=0.42), a finding not previously reported. The area under the ROC curve discriminating autism vs. NT using multiple gaze features was 0.90, 95% CI=(0.82, 0.97). We examined model performance using multiple gaze features by sex, race, and ethnicity. AUCs for iPhone and iPad, respectively: male: 0.87, 0.88; female: 0.88, 0.90; Black: 0.89, 0.71; white: 0.88, 0.91; Asian/other races: 0.88, 0.92; Hispanic/Latino: 0.91, 0.84. Results lack statistical confidence given the small sample sizes, however. Performance in males and females was comparable.

Preliminary results with children with developmental delay. In Study 2, among the children who were evaluated, a subgroup was diagnosed with developmental delay/language delay (DDLD). Descriptive data suggest that some digital phenotypes are specific to autism.¹³

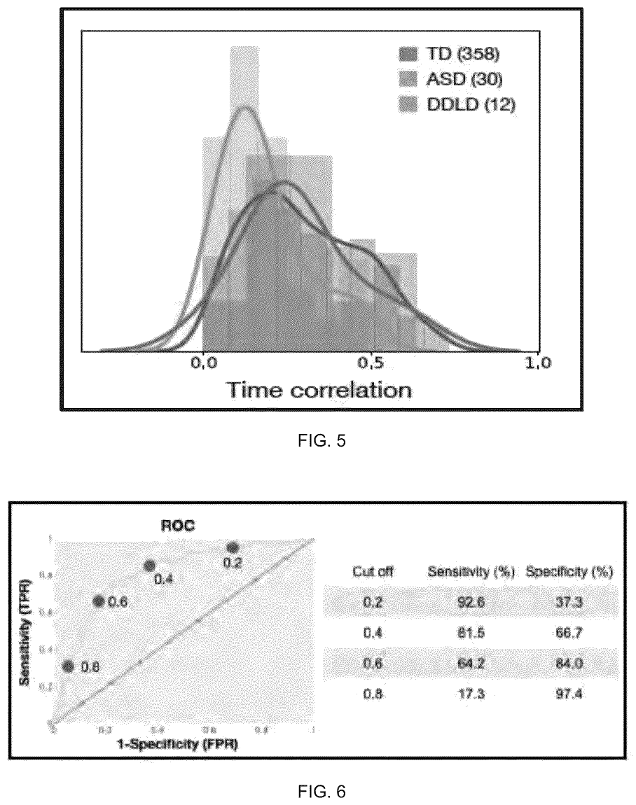

As shown in FIG. 5, social attention to speech by DDLD toddlers is more like NT children. We anticipate that some digital markers will overlap with other neurodevelopmental disorders and others will be specific to autism.

Performance based on combining more than one type of feature. The S2K-TP screening algorithm combines multiple features to provide a more robust early detection tool. As an initial step toward this goal, we assessed the sensitivity and specificity of S2K-TP, combining two response-to-name features (frequency and latency of head turn), two social attention features (percent right scores), and the speech-gaze correlation feature. As shown in FIG. 6, results are encouraging and better performance is expected as several more features are added (e.g., facial expressions and dynamics, postural sway, motor skills).

Test-retest reliability. We assessed test-retest reliability of CVA features in 102 autistic children who received a 2nd administration from 1 to 55 days later (M=1.04, SD=10.67). Within-subject test-retest reliability of response patterns over items (alpha statistic) was high (Mean rqq=0.99 [SD=0.03]; range=0.80-0.99). The interclass correlation (ICC) for individual CVA features ranged from 0.60-0.80. Days between visits in the tested range did not affect outcomes.

Evidence for convergent validity of S2K-TP. Based on data from 40 autistic children in Study 2, we examined correlations between ADOS calibrated symptom severity scores and both social attention and gaze-speech correlations derived during social attention movies. Total ADOS severity scores significantly correlated with both percent right and silhouette coefficients to ‘Blowing Bubbles’ (r=−0.27, P<0.05; r=−0.45, P<0.01) and ‘Spinning Top’ (r=−0.27, P<0.05; r=−0.31, P<0.05). Total ADOS severity score significantly correlated with gaze-speech coordination silhouette coefficient (‘Dyadic Conversation’: r=−0.52, P<0.01).

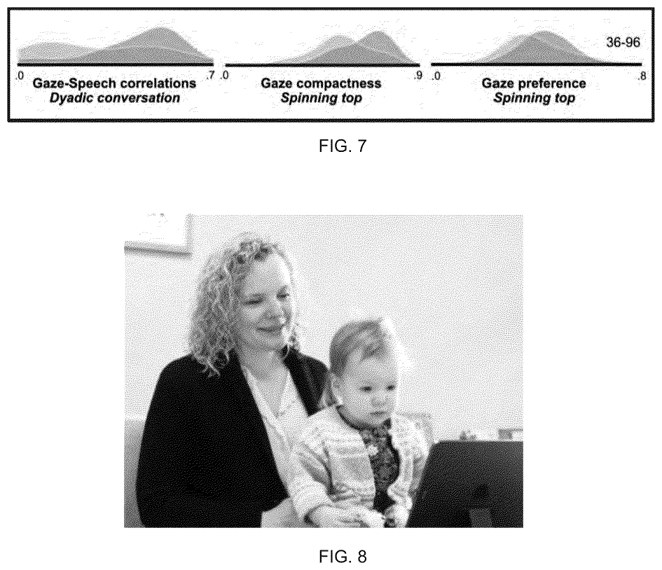

Preliminary evidence for usability of S2K-TP with older children. As part of our ACE, we also tested 76 3- to 8-year-old autistic and 26 age-matched NT children. Completion of the app was comparable to that of toddlers. The AUC using the combination of social attention gaze features showed differentiation between groups (AUC=0.76) suggesting that S2K will be useful for measuring autism-related behaviors in older children (see FIG. 7).

Evidence for feasibility of sample recruitment and remote administration of S2K. In our current ACE, we built a robust platform for recruiting a diverse, community-based sample of participants in partnership with Duke Primary Care. 6-month-old infants and their parents are recruited through Duke Primary Care clinics to evaluate an infant version of S2K which is being administered remotely and longitudinally by parents at home. To develop the capability of remote administration, we implemented a system for inviting parents' participation through the Duke Health electronic patient portal (MyChart). Using Twilio, an integrated system with REDCap, we implemented automated SMS reminders, prompts, and feedback for remote survey and app completion. We created multilingual instructions including animations and YouTube videos illustrating how to download and administer the app. Changes were made to the app itself, including a new back-end infrastructure for remotely uploading videos to Duke servers that meets security and privacy standards and considers the user's device type and uploading speed. Improvements to the user interface allow parents to download and navigate the app independently with automatic registration of the app and its data; no research assistant needs to be involved in the pipeline, although they are readily available to provide support by phone, text, and Zoom. To date, >630 parents have enrolled in the infant S2K study, which has yielded high quality videos for analysis. Our positive experience with this study parallels a previous feasibility study with remote administration at home of an early version of the app conducted on Apple's ResearchKit platform. Within one year, we enrolled 1,756 families of children 12-72 months, who completed 5,618 parent surveys and uploaded 441 videos with 90% usable data and demonstrated differences in affect and attention by autism status.⁴¹

### Data—Aim 3

Feasibility of remote telehealth recordings of parent-child interaction. During the current ACE, we developed a telehealth protocol (see below) for a 6-minute, parent-child interaction using toys/objects from the home and collected⁵⁵ parent-child interactions with young autistic children. Eight interactions were coded by three coders who were trained on 11 items from the Joint Engagement Rating Inventory (JERI).^{54, 98, 99} Fifty percent of the recordings were double coded by a master rater to determine agreement (rating within one point for each item). Percent agreement across the 11 codes were: Coder 1, 100%; Coder 2, 100%; Coder 3, 82% (average=91%). This demonstrates the ability to conduct and code parent-child interactions collected remotely. Preliminary results of CVA coding of lab-based parent-child interactions. Using 74 lab-based videos of parent-child interaction in children aged 41-100 months collected in the current ACE, we explored whether CVA could detect the temporal coordination between a child and parent with respect to initiating play by bending to reach for a toy during free play, which we labelled “synchrony.” CVA coded each instance of this behavior (“bending/reaching”) for the child and parent, and then examined whether reaching by one partner influenced reaching of the other partner. Dyadic data analysis methods¹⁰⁰ (Actor-Partner Interdependence Model, APIM100,101 and multi-level general linear models) were applied to time-series variables,¹⁰² yielding distinct transition probabilities (e.g., parent reaching was followed by child reaching). Autistic children who had higher Socialization and Communication Subscale Scores on the Vineland Adaptive Behavior Scales¹⁰³ displayed more synchronous interactions, reflecting an increased probability that the child would reach following parent reach (P<0.01 and 0.05, respectively). Child reaching was correlated with the total time spent in “joint engagement” coded by naïve coders using the JERI scale (P<0.05, r=0.25).⁵⁴ To test the accuracy of computer versus human coding of reaching for a toy, a set of videos was coded, noting each time the child and adult reached down to the floor to initiate toy play. Agreement between events of reaching to initiate toy play and CVA annotated reaching/bending was 0.77 for parent and 0.92 for the child. Using videos of parent-child interaction recorded via telehealth in our current ACE, we are determining optimal home recording conditions for CVA analysis.

### Data—Aim 4

Evidence for importance of a digital screening tool with automated decision support. In our current ACE, we conducted a retrospective EHR chart review for 213 children receiving care in the Duke Primary Care system who had received a positive M-CHAT-R/F. We found that Hispanic families experienced longer delays in receiving a referral (P<0.05), diagnostic evaluations (P=0.05), and starting services (P<0.01), and Black families experienced delays in starting services compared to white families (P<0.05, in preparation). Our results parallel previous findings of disparities in timing and access to services by families from Black and Hispanic/Latino backgrounds.^{61, 63} In a separate study, we evaluated the impact of combining a digital version of the M-CHAT—R/F with automated CDS which was integrated into the EHR (Epic) and provided to primary care providers during 18-24 month well-child visits.⁶⁵ The sample was 1,191 children 16-30 months old. After implementation of the CDS, provider rate of referrals for evaluation and/or services after positive screens increased from 25% to 85%. On an anonymous survey, most (90%) of physicians stated that the automated CDS improved their screening for autism. We then followed the study sample for 2 years by examining their EHR and found children whose provider used the CDS were 5 times more likely to be referred for evaluation and received an autism diagnosis 3.7 months earlier, on average.⁶⁶ These results suggest that a screening approach that includes automated decision support will increase rate of referrals and lower the mean age of diagnosis.

A longitudinal, prospective study of ˜5,000 participants recruited through four Duke Pediatric Primary Care Clinics that serve a diverse population of families who are both privately and publicly insured is also pursued. As the primary provider, and only hospitals in Durham County, NC, ˜85% of children in the county get care from Duke Health.¹⁰⁴ Durham is a diverse, majority non-white county with variation in demographic and socio-economic status. As part of routine pediatric care, children will receive the M-CHAT-R/F at their 18- and 24-month well child visits. Parents will be invited to participate via the web-based patient portal, MyChart, and can provide eConsent before the visit. During the visit, the parent or legal guardian will be approached by study staff embedded full time at the clinics. If the parent/guardian has not provided eConsent, they will be invited to participate and consent in person. Consent will include permission to collect information for the study from their child's EHR. Study and app download/instructions will be offered at the clinic visit. If the parent prefers, they will be offered an instructional brochure and/or a meeting via Zoom, or they can watch an instructional YouTube video in English or Spanish. To mitigate sample bias based on access to an iPhone, iPad, and/or broadband internet, we will have an iPad in each clinic and offer parents the option of completing the app and surveys during their clinic visit, and paper surveys can be sent by mail with return postage. We will collect systematic data on access to an iPhone/iPad and parent preferences regarding assessment modalities to understand the barriers and potential biases inherent to technology-based studies. All participants will be followed longitudinally with remote or in-clinic app and survey completion at 16-30, 36, and 48 months.

Defining diagnostic groups. The following procedures will be used to determine a diagnosis of autism spectrum disorder (ASD), DDLD, other disorder, and NT. First, participants who receive a positive score on the M-CHAT-R/F at their routine 18- or 24-month well-child visit or whose provider has a concern regarding autism will be offered a diagnostic evaluation by the study team (see details below). Based on this evaluation, children will receive a DSM-5 diagnosis of ASD, DDLD, “Other,” or NT. Second, in order to capture diagnoses for children who choose not to be evaluated by the study team, for all participants, we will record all neurodevelopmental and psychiatric diagnoses indicated in the EHR through age 4 years. Per Guthrie et al., children will be considered to have a diagnosis of ASD if an ICD-9/10 ASD diagnostic code (DSM/ICD codes 299, 299.8, F84.0, F84.5, F84.8, F84.9) appears more than once or is provided by an autism specialist (including our team).^{3, 105}

Thus, children can receive a diagnosis based on our team, or the EHR, or both. Consistent with Guthrie et al.,³ children with non-autism DDLD will be defined as >1 ICD code for developmental delay, language disorder and/or delay, motor disorder and/or delay, and social delay without autism. An EHR-based “Other” group will be comprised of children who received >1 ICD code specifying attention-deficit/hyperactivity disorder (ADHD) and related behaviors, anxiety disorder and related behaviors, and/or disruptive behavior disorder and related behaviors. Children will be considered NT who did not receive a positive score on the M-CHAT-R/F and who never received an autism or other non-autism disorder EHR diagnostic code.

Use of core resources. The Administrative Core will facilitate collaboration among all three Projects, oversee the overall budget and resources, interface with the IRB, establish and monitor QA standards, and monitor the combined progress of the Center. The Data Management and Analysis Core will facilitate high-quality data collection via REDCap, maintain the centralized database, establish and monitor data quality, store and clean data, assist with analysis of data, and assist with and track recruitment and retention, including diversity of participants, per our PEDP. The Dissemination and Outreach Core will promote two-way communication with the community; seek feedback about study feasibility, acceptability, and burden; promote junior and diverse investigators; and disseminate knowledge gained from this project to the community.

Rigor and reproducibility. Approaches in this Example are well-powered to test the hypotheses of the Project and allow meaningful estimates of the influences of race, ethnicity, sex, family income, and maternal education. Quality Assurance includes training on Standard Operating Procedures (SOPs), tracking SOP deviations, and implementation of Good Clinical Practice in all aspects of the Project. Research will be monitored by a Regulatory Coordinator who identifies areas of deficiency, aspects of GCP that need reinforcement, or additional training. Fidelity to procedures is accomplished by well-defined protocols, Manuals of Operation, reliability training, and internal audits. A diverse sample is recruited using a well-established partnership with Duke Primary Care clinics, and is implementing new strategies to increase diversity (See our PEDP and Dissemination and Outreach Core).

### Methods—Aims 1 and 2: Assess S2K-TP's Accuracy for Autism Detection and its Convergent Validity Compared to Clinical Measures

### Participants

Inclusion/exclusion criteria. Any child who is between 16-30 months of age who is presenting to the clinic for an 18- or 24-month well-child visit, and whose parent/legal guardian speaks English or Spanish and provides informed consent, will be invited to participate. Children who are coming to the clinic for a sick visit and/or have a vision, hearing, or motor impairment that preclude study measure completion will be excluded.

Ascertainment strategy and sample size estimates. To estimate anticipated sample size, we used the enrollment numbers from our current ACE study being conducted in primary care clinics. From January 2019-March 2021, we enrolled >3,000 families (˜111 per month). 293 children (9.7%) had a positive M-CHAT-R/F and/or their provider had a concern regarding autism and were referred for evaluation. Of those referred, 72% were diagnosed with autism spectrum disorder (ASD), and all but one of the remaining children were diagnosed with DDLD. In the proposed study, because the recruitment partnerships with primary care clinics are already established, we anticipate being able to collect data for 48 months. At ˜111 per month, the estimated number of enrolled participants=5,238. Based on prior experience, we expect 1/5 enrolled participants will not complete the app. We will have 4,190 children with app data, and of those, 406 (9.7%) will be referred based on either a positive M-CHAT-R/F or provider concern. Of those, 293 children (72%) are expected to be diagnosed with ASD (234 boys, 59 girls based on 4/1 ratio), and ˜116 children are expected to have a diagnosis of DDLD. To estimate the sample demographic distribution, we conservatively used the demographic characteristics of the sample of >3,000 families who are part of a current ACE project being conducted in the same primary care clinics. Comparing current ACE participant demographic percentages to those of Durham County, NC, we recruited a higher percentage of Hispanic (16.2% vs. 13.7%) and American Indian (2.5% vs. 0.9%) participants and those with more than one race (8.9% vs. 2.6%), and a lower percentage of Black participants (18.8% vs. 36.9%). Percentages for Asian and Hawaiian/Pacific Islander were similar. Our participant population is majority non-White (53.7%). Increasing enrollment of Black participants is a key goal for our proposed ACE per our PEDP.

### Clinical Measures for all Participants

Demographic information will be obtained via a brief survey, including birthdate, race, ethnicity, sex, family income, maternal education, and primary language. Insurance status and zip code will be extracted from the EHR. Medical history will be extracted from the EHR, including gestational age and weight at birth, birth and perinatal events, medical visits, diagnoses (e.g., seizures, GI), and treatment history (e.g., physical therapy). Other behavioral measures will include the following: As part of routine pediatric care, all children will be screened for autism at their 18- and/or 24-month well-child visit with the M-CHAT-R/F1 and for developmental concerns at their 18-, 24-, 36- and 48-month well-child visits with the Survey of Well-being of Young Children Milestones (SWYC),^{106, 107, 108} a developmental surveillance and screening tool with adequate psychometric properties.¹⁰⁹ At 16-30 months, we will administer the Child Behavior Checklist 1½-5 (CBCL),¹¹⁰ which has been widely used as a measure of behavioral functioning, including anxiety, depression, aggression, attention problems, emotional reactivity, somatic complaints, withdrawal, and sleep problems, and the Vineland Adaptive Behavior Scales, 3rd Ed (VABS-3) Parent/Caregiver Form¹⁰³ The VABS is a well-validated parent report of adaptive function, including T-scores for communication, daily living skills, socialization, motor skills, and challenging behavior. At 36- and 48-months, all participants will be administered the CBCL, VABS-3, and the Social Responsiveness Scale-2 (SRS-2) a parent survey which measures autism-related behaviors.^{111, 112}

Clinical Measures for Participants with Positive M-CHAT-R/F

Diagnostic evaluations. Children who score positive on the M-CHAT-R/F or whose provider has an autism concern at either the 18- or 24-month well-child visit will be offered a diagnostic and cognitive evaluation by the study team. A DSM-5 diagnosis of ASD will be based on information from the Autism Diagnostic Observation Scale (ADOS-2)⁹⁷ and the Autism Diagnostic Interview—Revised (ADI-R).¹¹³ If a parent is unable to travel to Duke for an in-person evaluation, they will be offered an evaluation via telehealth with the TELE-ASD-PEDS,¹¹⁴ appropriate through 36 months.¹¹⁵ Remote autism diagnostic evaluations have been shown to reduce disparities in receiving a diagnosis for under-resourced families.^{68, 115-119} The TELE-ASD-PEDS is conducted by via Zoom by an experienced, ADOS-reliable clinician who coaches the parent to engage in simple play activities involving requesting and social interaction. At 36- and 48-months, diagnostic evaluations will be repeated. If a parent is unable to travel to Duke for in-person ADOS evaluation, we will offer via telehealth the Brief Observation of Symptoms of Autism (BOSA)¹²⁰ developed by Catherine Lord, which involves observing parents engaging in semi-structured activities their child via Zoom. For the past 2 years, we have been successfully using the TELE-ASD-PEDS and BOSA for remote diagnostic evaluation in our research program and the Duke Autism Clinic (Dawson, Director). All evaluations will be conducted by an ADOS research-reliable licensed psychologist. As part of the diagnostic evaluation, children will also be assessed with the ACE Subject Medical History Questionnaire and the ACE Family Medical History Questionnaire.

Cognitive/developmental evaluation. At 16-30, 36, and 48 months, cognitive abilities will be assessed with the Developmental Assessment of Young Children-2nd Ed (DAYC-2),¹²¹ which is a normalized measure that can be administered in person or via telehealth to children birth-6 years, yielding standard scores, percentiles, and age equivalents for cognitive, communication/verbal, social-emotional abilities, and adaptive behavior. A cut-off of 90 has demonstrated excellent sensitivity for identification of developmental/language delay. 122

### S2K-TP App for all Participants

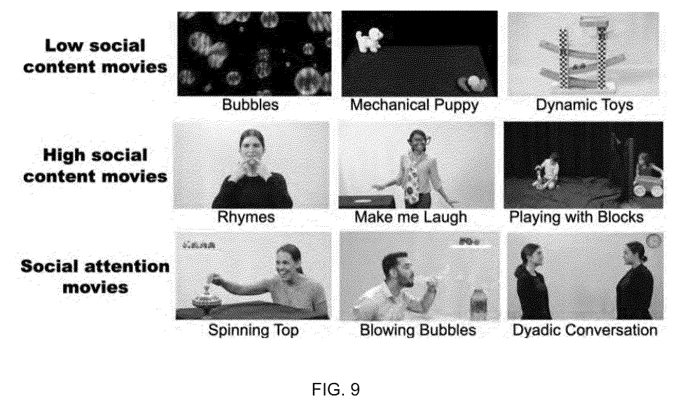

At 16-30, 36, and 48 months, parents of all participants will be asked to administer the S2K-TP app with their child. Parents of a subgroup of 100 autistic and 100 NT toddlers will be administered S2K-TP twice, one week apart, to assess test-retest reliability. No information from S2K-TP will be provided to parents and providers at any time during the study. Parents will be asked to sit their child on their lap in front of their own iPhone or iPad placed on a table in an upright position using books or a stand (See FIG. 8). We will offer to send a stand via mail to their home for them to keep. The instructions in the app ask that parents remain quiet and not direct their child's behavior or attention, except for the bubble popping game. The parent is asked to call the child's name 3 times when they see a small speaker icon embedded on the movies. Parents can pause the app by tapping the screen and resume for up to one hour. The frontal camera in the device records video throughout movies at 1280×720 resolution and 30 frames per second. S2K-TP stimuli consist of a series of 9 short movies (see FIG. 9), and a bubble-popping game, that in total take <10 minutes to administer. The videos are automatically and securely uploaded to Duke servers where staff perform review of the quality of app administration (for tracking and training purposes), and videos are processed through an automatic CV and ML analysis pipeline.

CVA of S2K videos. Each video will be processed through a face detection and recognition algorithm.¹²³ To ensure the face analyzed corresponds to the participant, a semi-supervised face-tracking algorithm will be used to verify a handful of low confidence frames. For each frame for which the participant is detected, facial landmarks, head pose, facial action units, and gaze will be extracted.^{77, 124-129} To combine all CVA features, we will exploit standard tools from ML, including random forests, XGBoost, and Gaussian mixture models (GMMs), which can handle missing information (e.g., missing attention to one of the movies) without explicit imputation. We will use data from the current sample of 993 children as the training data set, and the newly acquired data for testing, adding standard cross-validation and adaptation via transfer learning and domain shift as needed. We also plan to add new features related to vocalizations. Based on human coding of vocalizations and sounds made by toddlers while watching the app, we found that autistic toddlers displayed fewer consonant-vowel vocalizations than NT toddlers.¹³⁰ We will explore audio and CV analysis of facial/mouth movements to detect types of vocalizations.

### Methods—Aim 3: Explore the Feasibility of Using CVA to Measure Patterns of Parent-Child Interactions Videotaped at Home and its Convergent Validity Compared to Human Coding.

Participants. Fifty 16-30-month-old children with a diagnosis of ASD based on the ADOS-2 and ADI-R and 50 age- and sex-matched NT children will be recruited from the larger study population for the feasibility study using CVA to measure parent-child interactions. Inclusion criteria include age and diagnosis of ASD or NT as defined above.

Exclusion criteria include (1) known genetic or neurological syndrome, (2) motor or sensory impairment that would interfere with valid completion of study measures, and (3) untreated epilepsy.

### Measures

Recording parent-child interactions via telehealth. Parent-child interactions will be recorded using the parent's personal recording device and Zoom technology. Whereas for the S2K-TP app we will offered in-person administration at the clinic, thereby mitigating sample bias due to lack of an iPhone or iPad, we recognize that use of Zoom will bias our sample since some parents will not have a recording device or want to use Zoom. Parents will be coached to find toys/objects in specific categories and an optimal place for recording. Camera angles will be checked to position the recording device to capture the parent and child, including hands and activities/toys, with adequate lighting. Parents and children will be asked to play naturally for 6 minutes.

CVA methods for coding parent-child interactions. CVA results will be acquired with an existing automated pipeline for video data processing built from open-source tools. For synchrony, 2D pose estimation¹³¹ will provide 2D skeleton landmarks and confidence scores for each landmark and index data quality. Person detection¹³² and in-house tracking code will allow separation of parent and child. 3D pose estimation¹³³ will provide landmarks in

3D space with respect to the camera coordinate system. Metrics for torso movement in the vertical plane (‘reaching’) will be extracted from 3D landmarks and post-processed using in-house code, resulting in a binary time-series signal per each person with 1 Hz discretization, where ‘1’ was ‘person reached’ and ‘0’-‘person did not reach’. A pair of time-series will then be transformed into a single time-series with four states. Then dyadic data analysis methods (APIM and multi-level general linear models^{100, 101}) will be applied to the time-series data.¹⁰² Proximity will be extracted as a physical distance between “base of the neck” landmarks of the parent and child in 3D space. Orientation will be computed as the angle in horizontal plane (parallel to the floor), between torso directions (ToD) of parent and child. ToD will be computed as a normal vector to the torso plane (cross product of the left-to-right shoulder axis and neck-pelvis axis) and projected onto the horizontal plane.

Human coding of parent-child interactions. To code synchrony, we will use the following 7-point ratings of items from the JERI coding system: (a) Dyadic Fluency and Connectedness (quality of connectiveness); (b) Child Responsiveness to Partner's Communication (responses to adult communication bids); and (c) Joint Engagement (time spent in a shared activity).⁹⁸ To code child orientation and proximity, we will use Child Attention to Parent (quantity and quality of the child's explicit attention to and involvement with the parent). We will also code discrete behaviors that represent synchrony, proximity and orientation such as the durations and frequencies of bending and reaching to a shared object or activity, and time spent facing each other. No feedback about the CVA or human coding of parent-child interactions will be provided to parents.

### Methods—Aim 4: Design and Assess the Perceived Usability of a CDS for Autism Screening and Define a List of Key Priority Factors for Using an Autism Screening CDS Across a Broad Range of Settings.

Design Process. The design process involves active, longitudinal engagement to both design a CDS tool prototype refined through iterative feedback at multiple time points and identify key priority factors to consider when designing and implementing CDS broadly. The CDS design process occurs in two phases. First, we will evaluate end-user contexts to understand user characteristics and tasks; the physical, technical and organizational environment; and other preferences. 134 To understand end-user contexts, two groups of PCPs will be recruited using purposeful sampling, one group from within DUHS (n=10) and one group representing community practices (n=10).135 We will conduct qualitative individual interviews with providers through Zoom or in-person with a focus on environmental factors, such as work flow constraints, and roles and responsibilities of the care team.¹³⁴

Interviews will be audio recorded, transcribed verbatim, and analyzed using directed content analysis.¹³⁶ We will aggregate coded data from the academic and community PCP groups to compare data by setting. Data will be stored, managed, and queried with qualitative data analysis software, NVivo¹². Also, for the DUHS PCPs only, we will conduct observations in the clinical care environments, shadowing them to understand the details of their clinical workflows and environmental contexts. End-user contextual information gained from the PCP groups will be integrated through qualitative analysis and fed back in a simple visual format to inform the next phase—prototype development. In human-centered design, ease of use (usability) is the primary outcome of interest and a key determinant of implementation outcomes.¹³⁷ For usability assessment, a new group of DUHS PCPs (n=20) will be recruited using purposeful sampling techniques.¹³⁵ In groups of 5, the PCPs will be shown a CDS prototype and complete the System Usability Scale.¹³⁸ Responses on this scale will inform an interview guide, which will be used in focus group discussion with the PCPs that will increase understanding of usability constructs. An iterative process of feedback and refinements continues through 3-4 cycles (˜5 PCPs per cycle) until PCPs' scores on the System Usability Scale are consistently rated as ‘acceptable’ and qualitative data support this finding. Note that this study will use stakeholder feedback to design the CDS prototype but will not implement or evaluate use of CDS in primary care.

List of Priority Factors. Using information gained through two phases of the design process, a list of key priority factors to consider when designing an autism screening CDS system is compiled.

### Statistical Approaches and Power Analyses

Analyses will be conducted in collaboration with the Data Management and Analysis Core. Descriptive statistics and graphical summaries will screen for outliers and inform transformations or nonparametric methods.

Aim 1. To assess the accuracy of S2K-TP for autism detection, the primary analyses will be ML classification algorithms. We will frame the analyses as ML with class imbalance.¹³⁹ Standard ML performance metrics are suboptimal for evaluating classification methods when prevalence rates are low (e.g., autism).^{140, 141-143} Python libraries are used for ML analysis including scikit-learn to test classifiers, XGBoost, SVM, and GMM. Imbalanced-learn are used to implement strategies specific of imbalanced problems.^{144, 145} Algorithm performance is assessed using Precision-Recall and ROC curves. Cross-validation is used to estimate sensitivity, specificity, and negative/positive predictive value, with the sample being sequentially separated into a training and testing set. The former will be used to fit classification models, while performance is reported on the latter. We will also use of the current preliminary data for training and the new data in this project for testing. SMOTE and other algorithms suited for imbalanced problems will also be considered.¹⁴⁶⁻¹⁴⁸ Confidence intervals will be estimated using the bootstrap method.149, 150 Test-retest analyses will be conducted on a subsample of 100 NT and 100 autistic children. We will use the psych R package functions to calculate test-retest reliability.¹⁵¹

Intraclass correlation coefficients (ICC) will be calculated following empirically established guidelines.¹⁵² Mixed effects/multilevel modeling is well known for examining within-person effects across time, and the Ime4 and nIme R packages are well-equipped to examine longitudinal app performance.^{153, 154} To examine the influence of race, ethnicity, sex, family income, and maternal education on the autism prediction accuracy, we will compare accuracy, sensitivity, and specificity across these subgroups.

Aim 2. To assess convergent validity of S2K-TP at different ages, the resulting ML classification fitted value will be compared to standardized clinical measures of autism-related behaviors. For social function, we will use the VABS Socialization Subscale Score and SRS-2 Social Motivation Subscale. Language and motor abilities will be assessed via the VABS Communication and Motor Subscales. First, ML output will be correlated with the clinical measures. The classifier will then be entered into factor analytic methods. Component parts of the CVA variables are hypothesized to relate to specific clinical outcomes, and thus confirmatory factor analytic (CFA) methods will test those structural hypotheses. CFA will be conducted with the sem and lavaan R packages.^{155, 156} Regarding concurrent and longitudinal relationships, multilevel models will be computed where time points will be nested within children. Moderating effects of diagnostic group membership on CVA measure relationship across time will be examined. The Ime4 and nIme R packages are suited for this multilevel modeling.

Aim 3. To assess the feasibility of using CVA to measure parent-child interactions from videos taken at home, we will first determine the amount of usable data for autistic and NT toddlers. To examine convergent validity of CVA measures of parent-child interaction, the subset of 50 NT and 50 autistic children will be analyzed with time-series, multilevel, dyadic analyses which will be applied to derived CVA measures of synchrony, proximity, and social orientation. For synchrony, dyadic data analysis methods¹⁰⁰ (APIM100, 101 and multi-level general linear models) will be applied,¹⁰² yielding distinct transition probabilities. Correlations between the transition probabilities and standardized and coded measures of social reciprocity (VABS Socialization subscale and joint engagement ratings on the JERI scale) will be calculated. For proximity and social orientation, correlations between CVA-derived measures and human coding of these constructs will be calculated.

Aim 4. To assess the perceived usability of a CDS tool that includes actionable guidance, explanatory sequential mixed methods design will be used.¹⁵⁷ Results from the System Usability Scale¹³⁸ will indicate that end-user usability scores are consistently rated as ‘acceptable’ and qualitative data will support this finding.

### Power Analyses—Power Analyses Utilize GPower158 and Optimal Design.¹⁵⁹

Aim 1. A precise calculation of sensitivity and specificity for S2K-TP requires larger samples when the prevalence of the target group is low (i.e., the group with autism).¹⁶⁰⁻¹⁶² For Aim 1 we require at least N=980 (participants) and n=49 (diagnosed with autism).¹⁶⁰ The proposed sample size will be more than adequate to properly calculate sensitivity and specificity for S2K-TP. Test-retest calculations require at least N=50; greater than N=100 is recommended,¹⁶³ which our projected subsample exceeds. Lower-bound recommendations for multilevel analyses include a general rule of 3 measurements at the lowest level and 30 participants at the higher level¹⁶⁴. Examining multilevel models within autistic children will be adequately powered. Testing demographic differences in ML will follow the guidelines for sensitivity and specificity mentioned above,¹⁶⁰ meaning any a Demographic differences post-ML will be more than adequately powered if conducted in the full sample. Diagnostic by demographic interactions would be underpowered in some groups (e.g., Asian autistic children). Expected sex differences (one-tailed tests) within autistic children would be detected with effect sizes Cohen's d>0.36, while two-tailed tests would require Cohen's d-to-large effects would be required to test within-diagnostic group differences by race and/or ethnicity. We do not expect to test DDLD within-group differences without binarizing variables.

Aim 2. To evaluate convergent validity of S2K-TP, bivariate correlations within autistic children (n=293) will be detected at r level analyses will be able to detect minimal effect sizes with adequate power. Confirmatory factor analyses (CFA) have many sample size considerations.¹⁶⁵ Analyses with both the full sample and subsample of autistic children meet these considerations.

Aim 3. To evaluate convergent validity of CVA coding of parent-child interaction, importantly, this subset of 100 children will have CVA data that is broken down into 0.5 or 1.0 second intervals. Preliminary data gave an average of 500 time points per participant, thus an expected N=50,000 data points. Multilevel repeated measure power calculations yield that longitudinal unstandardized effects of B children, and B suggests a minimum of N=35 dyads, and from his calculations we should be able to detect nonindependence in dyads via intra-dyad correlations above r=0.3.¹⁰⁰

Aim 4. This aim is primarily qualitative and implementation-based, focusing on the accepted usability by providers, caregivers, and stakeholders. There are no parametric tests proposed.

Potential Difficulties and Limitations and how these are Overcome or Mitigated

Although our past experience indicates that we will be able to recruit a diverse sample (53.7% non-white), our is to examine performance of S2K-TP based on demographic factors. We plan to implement several new strategies to increase recruitment of Black families (see Recruitment and Retention Plan in Overall and PEDP). If we identify challenges in reaching our recruitment goals, we have an existing strong collaboration with four additional Duke Primary Care clinics who are participating in our current infant autism screening study and will explore further recruitment strategies with consultation from the Dissemination and Outreach Core.

Future studies include (1) examining the use of the digital tools developed in this project for outcome monitoring in the context of an early intervention trial, (2) conducting an intervention study using S2K with CDS in a primary care setting to examine its impact on referral rates, age of autism diagnosis, and initiation of services; and (3) extending the use of a digital phenotyping tool for school-age and adolescent autistic children and autistic adults.

### REFERENCE CITED IN EXAMPLE

The disclosure of each of the following references is incorporated herein by reference in its entirety to the extent that it is not inconsistent herewith and to the extent that it supplements, explains, provides a background for, or teaches methods, techniques, and/or systems employed herein.
1. Robins D L, Casagrande K, Barton M, Chen C M, Dumont-Mathieu T, Fein D. Validation of the modified checklist for Autism in toddlers, revised with follow-up (M-CHAT-R/F). Pediatrics. 2014; 133(1):37-45. PMID: 24366990; PMCID: PMC3876182.
2. Dawson G. Why It's Important to Continue Universal Autism Screening While Research Fully Examines Its Impact. JAMA Pediatr. 2016; 170(6):527-8. PMID: 26882277.
3. Guthrie W, Wallis K, Bennett A, Brooks E, Dudley J, Gerdes M, Pandey J, Levy S E, Schultz R T, Miller J S. Accuracy of Autism Screening in a Large Pediatric Network. Pediatrics. 2019; 144(4). PMID: 31562252.
4. Scarpa A, Reyes N M, Patriquin M A, Lorenzi J, Hassenfeldt T A, Desai V J, Kerkering K W. The Modified Checklist for Autism in Toddlers: Reliability in a diverse rural American sample. J Autism Dev Disord. 2013; 43(10):2269-79. PMID: 23386118.
5. Donohue M R, Childs A W, Richards M, Robins D L. Race influences parent report of concerns about symptoms of autism spectrum disorder. Autism. 2019; 23(1):100-11. PMID: 29100475; PMCID: PMC5930138.
6. Dickerson A S, Rahbar M H, Pearson D A, Kirby R S, Bakian A V, Bilder D A, Harrington R A, Pettygrove S, Zahorodny W M, Moyé L A, 3rd, Durkin M, Slay Wingate M. Autism spectrum disorder reporting in lower socioeconomic neighborhoods. Autism. 2017; 21(4):470-80. PMID: 27627912.
7. Maenner M J, Shaw K A, Baio J, Washington A, Patrick M, DiRienzo M, Christensen D L, Wiggins L D, Pettygrove S, Andrews J G, Lopez M, Hudson A, Baroud T, Schwenk Y, White T, Rosenberg C R, Lee L C, Harrington R A, Huston M, Hewitt A, Esler A, Hall-Lande J, Poynter J N, Hallas-Muchow L, Constantino J N, Fitzgerald R T, Zahorodny W, Shenouda J, Daniels J L, Warren Z, Vehorn A, Salinas A, Durkin M S, Dietz P M. Prevalence of Autism Spectrum Disorder Among Children Aged 8 Years-Autism and Developmental Disabilities Monitoring Network, 11 Sites, United States, 2016. MMWR Surveill Summ. 2020; 69(4):1-12. PMID: 32214087; PMCID: PMC7119644
8. McDonnell C G, DeLucia E A, Hayden E P, Penner M, Curcin K, Anagnostou E, Nicolson R, Kelley E, Georgiades S, Liu X, Stevenson R A. Sex Differences in Age of Diagnosis and First Concern among Children with Autism Spectrum Disorder. J Clin Child Adolesc Psychol. 2020; November 2(Epub ahead of print):1-11. PMID: 33136459.
9. Dawson G, Sapiro G. Potential for Digital Behavioral Measurement Tools to Transform the Detection and Diagnosis of Autism Spectrum Disorder. JAMA Pediatr. 2019; 173(4):305-6. PMID: 30715131; PMCID: PMC7112503.
10. Perochon S, Di Martino M, Aiello R, Baker J, Carpenter K, Chang Z, Compton S, Davis N, Eichner B, Espinosa S, Flowers J, Franz L, Gagliano M, Harris A, Howard J, Kollins S H, Perrin E M, Krishnappa Babu P R, Spanos M, Walter B, Sapiro G, Dawson G. A scalable computational approach to assessing response to name in toddlers with autism. J Child Psychol Psychiatry. 2021; 62(9):1120-31. PMID: 33641216; PMCID: PMC8397798.
11. Carpenter K L H, Hashemi J, Campbell K, Lippmann S J, Baker J P, Egger H L, Espinosa S, Vermeer S, Sapiro G, Dawson G. Digital Behavioral Phenotyping Detects Atypical Pattern of Facial Expression in Toddlers with Autism. Autism Res. 2021; 14(3):488-99. PMID: 32924332; PMCID: PMC7920907.
12. Dawson G, Campbell K, Hashemi J, Lippmann S J, Smith V, Carpenter K, Egger H, Espinosa S, Vermeer S, Baker J, Sapiro G. Atypical postural control can be detected via computer vision analysis in toddlers with autism spectrum disorder. Sci Rep. 2018; 8(1):17008. PMID: 30451886; PMCID: PMC6242931.
13. Chang Z, Di Martino J M, Aiello R, Baker J, Carpenter K, Compton S, Davis N, Eichner B, Espinosa S, Flowers J, Franz L, Harris A, Howard J, Perochon S, Perrin E M, Krishnappa Babu P R, Spanos M, Sullivan C, Walter B K, Kollins S H, Dawson G, Sapiro G. Computational Methods to Measure Patterns of Gaze in Toddlers With Autism Spectrum Disorder. JAMA Pediatr. 2021; 175(8):827-36. PMID: 33900383; PMCID: PMC8077044.
14. Rogge N, Janssen J. The Economic Costs of Autism Spectrum Disorder: A Literature Review. J Autism Dev Disord. 2019; 49(7):2873-900. PMID: 30976961.
15. Cidav Z, Munson J, Estes A, Dawson G, Rogers S, Mandell D. Cost Offset Associated With Early Start Denver Model for Children With Autism. J Am Acad Child Adolesc Psychiatry. 2017; 56(9):777-83. PMID: 28838582; PMCID: PMC7007927.
16. Estes A, Vismara L, Mercado C, Fitzpatrick A, Elder L, Greenson J, Lord C, Munson J, Winter J, Young G, Dawson G, Rogers S. The impact of parent-delivered intervention on parents of very young children with autism. J Autism Dev Disord. 2014; 44(2):353-65. PMID: 23838727; PMCID: PMC3888483.
17. Estes A, Munson J, Rogers S J, Greenson J, Winter J, Dawson G. Long-Term Outcomes of Early Intervention in 6-Year-Old Children with Autism Spectrum Disorder. J Am Acad Child Adolesc Psychiatry. 2015; 54(7):580-7. PMID: 26088663; PMCID: PMC4475272.
18. IACC. Interagency Autism Coordinating Committee Strategic Plan for Autism Spectrum Disorder (2018-2019 Update).
19. Johnson C P, Myers S M. Identification and evaluation of children with autism spectrum disorders. Pediatrics. 2007; 120(5):1183-215. PMID: 17967920.
20. Carbone P S, Campbell K, Wilkes J, Stoddard G J, Huynh K, Young P C, Gabrielsen T P. Primary Care Autism Screening and Later Autism Diagnosis. Pediatrics. 2020; 146(2):e20192314. PMID: 32632024; PMCID: PMC7397730.
21. Stenberg N, Bresnahan M, Gunnes N, Hirtz D, Hornig M, Lie K K, Lipkin W I, Lord C, Magnus P, Reichborn-Kjennerud T, Schjølberg S, Surén P, Susser E, Svendsen B K, von Tetzchner S, Oyen A S, Stoltenberg C. Identifying children with autism spectrum disorder at 18 months in a general population sample. Paediatr Perinat Epidemiol. 2014; 28(3):255-62. PMID: 24547686; PMCID: PMC3976700.
22. Sturner R, Howard B, Bergmann P, Stewart L, Afarian T E. Comparison of Autism Screening in Younger and Older Toddlers. J Autism Dev Disord. 2017; 47(10):3180-8. PMID: 28733850; PMCID: PMC5711534.
23. Yuen T, Penner M, Carter M T, Szatmari P, Ungar W J. Assessing the accuracy of the Modified Checklist for Autism in Toddlers: a systematic review and meta-analysis. Dev Med Child Neurol. 2018; 60(11):1093-100. PMID: 29992541.
24. Wetherby A M, Guthrie W, Hooker J L, Delehanty A, Day T N, Woods J, Pierce K, Manwaring S S, Thurm A, Ozonoff S, Petkova E, Lord C. The Early Screening for Autism and Communication Disorders: Field-testing an autism-specific screening tool for children 12 to 36 months of age. Autism. 2021; 25(7):2112-23. PMID: 33962531.
25. Allison C, Matthews F E, Ruta L, Pasco G, Soufer R, Brayne C, Charman T, Baron-Cohen S. Quantitative Checklist for Autism in Toddlers (Q-CHAT). A population screening study with follow-up: the case for multiple time-point screening for autism. BMJ Paediatr Open. 2021; 5(1):e000700. PMID: 34131593; PMCID: PMC8166626.
26. Pileggi M L, Brane N, Bradshaw J, Delehanty A, Day T, McCracken C, Stapel-Wax J, Wetherby A M. Early Observation of Red Flags in 12-Month-Old Infant Siblings Later Diagnosed With Autism Spectrum Disorder. Am J Speech Lang Pathol. 2021; 30(4):1846-55. PMID: 33989505.
27. McCracken J T, Anagnostou E, Arango C, Dawson G, Farchione T, Mantua V, McPartland J, Murphy D, Pandina G, Veenstra-VanderWeele J, Group IEAW. Drug development for Autism Spectrum Disorder (ASD): Progress, challenges, and future directions. Eur Neuropsychopharmacol. 2021; 48:3-31. PMID: 34158222.
28. Dawson G. Assessment of outcomes in autism clinical trials over the course of development. Eur Neuropsychopharmacol. 2021; 48:40-1. PMID: 33731267; PMCID: PMC8238809.
29. Anagnostou E, Jones N, Huerta M, Halladay A K, Wang P, Scahill L, Horrigan J P, Kasari C, Lord C, Choi D, Sullivan K, Dawson G. Measuring social communication behaviors as a treatment endpoint in individuals with autism spectrum disorder. Autism. 2015; 19(5):622-36. PMID: 25096930.
30. Scahill L, Aman M G, Lecavalier L, Halladay A K, Bishop S L, Bodfish J W, Grondhuis S, Jones N, Horrigan J P, Cook E H, Handen B L, King B H, Pearson D A, McCracken J T, Sullivan K A, Dawson G. Measuring repetitive behaviors as a treatment endpoint in youth with autism spectrum disorder. Autism. 2015; 19(1):38-52. PMID: 4259748.
31. Hansen R L, Ozonoff S, Krakowiak P, Angkustsiri K, Jones C, Deprey L J, Le D N, Croen L A, Hertz-Picciotto I. Regression in autism: prevalence and associated factors in the CHARGE Study. Ambul Pediatr. 2008; 8(1):25-31. PMID: 18191778.
32. Ozonoff S, Iosif A M, Baguio F, Cook I C, Hill M M, Hutman T, Rogers S J, Rozga A, Sangha S, Sigman M, Steinfeld M B, Young G S. A prospective study of the emergence of early behavioral signs of autism. J Am Acad Child Adolesc Psychiatry. 2010; 49(3):256-66 e1-2. PMID: 20410715; PMCID: PMC2923050.
33. Shumway S, Thurm A, Swedo S E, Deprey L, Barnett L A, Amaral D G, Rogers S J, Ozonoff S. Brief report: symptom onset patterns and functional outcomes in young children with autism spectrum disorders. J Autism Dev Disord. 2011; 41(12):1727-32. PMID: 21360021; PMCID: PMC3265105.
34. Ozonoff S, Iosif A M, Young G S, Hepburn S, Thompson M, Colombi C, Cook I C, Werner E, Goldring S, Baguio F, Rogers S J. Onset patterns in autism: correspondence between home video and parent report. J Am Acad Child Adolesc Psychiatry. 2011; 50(8):796-806 e1. PMID: 21784299; PMCID: PMC3668453.
35. Jones R M, Carberry C, Hamo A, Lord C. Placebo-like response in absence of treatment in children with autism. Autism Res. 2017; 10(9):1567-72. PMID: 28401674.
36. Thoma J, Bighelli I, Krause M, Rodolico A, Ceraso A, Deste G, Huhn M, Fraguas D, Mavridis D, Charman T, Murphy D G, Parellada M, Arango C, Leucht S. Placebo response in pharmacological and dietary supplement trials of autism spectrum disorder (ASD): systematic review and meta-regression analysis. Mol Autism. 2020; 11(1):66. PMID: 32847616; PMCID: PMC7448339.
37. Hsin H, Fromer M, Peterson B, Walter C, Fleck M, Campbell A, Varghese P, Califf R. Transforming Psychiatry into Data-Driven Medicine with Digital Measurement Tools. NPJ Digit Med. 2018; 1:37. PMID: 31304319; PMCID: PMC6550182.
38. Hirschtritt M E, Insel T R. Digital Technologies in Psychiatry: Present and Future. Focus (Am Psychiatr Publ). 2018; 16(3):251-8. PMID: 31975919; PMCID: PMC6493099.
39. Dawson G, Carpenter K, Chang Z, Di Martino J, Espinosa S, Perochon S, Krishnappa Babu P, Sapiro G. Computational approaches to the assessment of early motor behavior in autism. International Society for Autism Research Annual Meeting. 2021; May 3-7, 2021 (Virtual).
40. Campbell K, Carpenter K L, Hashemi J, Espinosa S, Marsan S, Borg J S, Chang Z, Qiu Q, Vermeer S, Adler E, Tepper M, Egger H L, Baker J P, Sapiro G, Dawson G. Computer vision analysis captures atypical attention in toddlers with autism. Autism. 2019; 23(3):619-28. PMID: 29595333; PMCID: PMC6119515.
41. Egger H L, Dawson G, Hashemi J, Carpenter K L H, Espinosa S, Campbell K, Brotkin S, Schaich-Borg J, Qiu Q, Tepper M, Baker J P, Bloomfield R A, Jr., Sapiro G. Automatic emotion and attention analysis of young children at home: a ResearchKit autism feasibility study. NPJ Digit Med. 2018; 1:20. PMID: 31304303; PMCID: PMC6550157.
42. Krishnappa Babu P R, Di Martino J M, Chang Z, Perochon S, Carpenter K, Compton S, Espinosa S, Dawson G, Sapiro G. Exploring complexity of facial dynamics in autism spectrum disorder. IEEE Translations on Affective Computing. In press.
43. Chawarska K, Macari S, Shic F. Decreased spontaneous attention to social scenes in 6-month-old infants later diagnosed with autism spectrum disorders. Biol Psychiatry. 2013; 74(3):195-203. PMID: 23313640; PMCID: PMC3646074.
44. Jones W, Klin A. Attention to eyes is present but in decline in 2-6-month-old infants later diagnosed with autism. Nature. 2013; 504(7480):427-31. PMID: 24196715; PMCID: PMC4035120.
45. Frazier T W, Coury D L, Sohl K, Wagner K E, Uhlig R, Hicks S D, Middleton F A. Evidence-based use of scalable biomarkers to increase diagnostic efficiency and decrease the lifetime costs of autism. Autism Res. 2021; 14(6):1271-83. PMID: 33682319; PMCID: PMC8251791.
46. Chita-Tegmark M. Social attention in ASD: A review and meta-analysis of eye-tracking studies. Res Dev Disabil. 2016; 48:79-93. PMID: 26547134.
47. Klin A, Jones W, Schultz R, Volkmar F, Cohen D. Defining and quantifying the social phenotype in autism. Am J Psychiatry. 2002; 159(6):895-908. PMID: 12042174.
48. Giarelli E, Wiggins L D, Rice C E, Levy S E, Kirby R S, Pinto-Martin J, Mandell D. Sex differences in the evaluation and diagnosis of autism spectrum disorders among children. Disability and health journal. 2010; 3(2):107-16. PMID: 21122776; PMCID: PMC4767258.
49. Lai M C, Szatmari P. Sex and gender impacts on the behavioural presentation and recognition of autism. Curr Opin Psychiatry. 2020; 33(2):117-23. PMID: 31815760.
50. Gotham K, Risi S, Dawson G, Tager-Flusberg H, Joseph R, Carter A, Hepburn S, McMahon W, Rodier P, Hyman S L, Sigman M, Rogers S, Landa R, Spence M A, Osann K, Flodman P, Volkmar F, Hollander E, Buxbaum J, Pickles A, Lord C. A replication of the Autism Diagnostic Observation Schedule (ADOS) revised algorithms. J Am Acad Child Adolesc Psychiatry. 2008; 47(6):642-51. PMID: 18434924.
51. Rogers S J, Estes A, Lord C, Vismara L, Winter J, Fitzpatrick A, Guo M, Dawson G. Effects of a brief Early Start Denver Model (ESDM)-based parent intervention on toddlers at risk for autism spectrum disorders: a randomized controlled trial. J Am Acad Child Adolesc Psychiatry. 2012; 51(10):1052-65. PMID: 23021480; PMCID: PMC3487718.
52. Kasari C. Assessing change in early intervention programs for children with autism. J Autism Dev Disord. 2002; 32(5):447-61. PMID: 12463519.
53. Schreibman L, Dawson G, Stahmer A C, Landa R, Rogers S J, McGee G G, Kasari C, Ingersoll B, Kaiser A P, Bruinsma Y, McNerney E, Wetherby A, Halladay A. Naturalistic Developmental Behavioral Interventions: Empirically Validated Treatments for Autism Spectrum Disorder. J Autism Dev Disord. 2015; 45(8):2411-28. PMID: 25737021; PMCID: PMC4513196.
54. Adamson L B, Bakeman R, Deckner D F, Nelson P B. Rating parent-child interactions: joint engagement, communication dynamics, and shared topics in autism, Down syndrome, and typical development. J Autism Dev Disord. 2012; 42(12):2622-35. PMID: 22466689; PMCID: PMC3445743.
55. Kojovic N, Natraj S, Mohanty S P, Maillart T, Schaer M. Using 2D video-based pose estimation for automated prediction of autism spectrum disorders in young children. Sci Rep. 2021; 11(1):15069. PMID: 34301963; PMCID: PMC8302646.
56. Moore G, Wilding H, Gray K, Castle D. Participatory Methods to Engage Health Service Users in the Development of Electronic Health Resources: Systematic Review. J Particip Med. 2019; 11(1):e11474. PMID: 33055069; PMCID: PMC7434099.
57. Lyon A R, Dopp A R, Brewer S K, Kientz J A, Munson S A. Designing the Future of Children's Mental Health Services. Adm Policy Ment Health. 2020; 47(5):735-51. PMID: 32253634; PMCID: PMC7395914.
58. Lyon A R, Munson S A, Renn B N, Atkins D C, Pullmann M D, Friedman E, Areán P A. Use of Human-Centered Design to Improve Implementation of Evidence-Based Psychotherapies in Low-Resource Communities: Protocol for Studies Applying a Framework to Assess Usability. JMIR Res Protoc. 2019; 8(10):e14990. PMID: 31599736; PMCID: PMC6819011.
59. Monteiro S A, Dempsey J, Berry L N, Voigt R G, Goin-Kochel R P. Screening and Referral Practices for Autism Spectrum Disorder in Primary Pediatric Care. Pediatrics. 2019; 144(4). PMID: 31515298.
60. Blanche E I, Diaz J, Barretto T, Cermak S A. Caregiving Experiences of Latino Families With Children With Autism Spectrum Disorder. Am J Occup Ther. 2015; 69(5):6905185010p1-11. PMID: 26356658.
61. Magaña S, Lopez K, Aguinaga A, Morton H. Access to diagnosis and treatment services among Latino children with autism spectrum disorders. Intellect Dev Disabil. 2013; 51(3):141-53. PMID: 23834211.
62. Smith K A, Gehricke J G, Iadarola S, Wolfe A, Kuhlthau K A. Disparities in Service Use Among Children With Autism: A Systematic Review. Pediatrics. 2020; 145(Suppl 1): S35-s46. PMID: 32238530.
63. Wallis K E, Guthrie W, Bennett A E, Gerdes M, Levy S E, Mandell D S, Miller J S. Adherence to screening and referral guidelines for autism spectrum disorder in toddlers in pediatric primary care. PLOS One. 2020; 15(5):e0232335. PMID: 32379778; PMCID: PMC7205236.
64. Kuhn J, Levinson J, Udhnani M D, Wallis K, Hickey E, Bennett A, Fenick A M, Feinberg E, Broder-Fingert S. What Happens After a Positive Primary Care Autism Screen Among Historically Underserved Families? Predictors of Evaluation and Autism Diagnosis. J Dev Behav Pediatr. 2021; 42(7):515-23. PMID: 33631787; PMCID: PMC8380258.
65. Campbell K, Carpenter K L H, Espinosa S, Hashemi J, Qiu Q, Tepper M, Calderbank R, Sapiro G, Egger H L, Baker J P, Dawson G. Use of a Digital Modified Checklist for Autism in Toddlers-Revised with Follow-up to Improve Quality of Screening for Autism. J Pediatr. 2017; 183:133-9.e1. PMID: 28161199; PMCID: PMC5397992.
66. Major S, Campbell K, Espinosa S, Baker J P, Carpenter K L, Sapiro G, Vermeer S, Dawson G. Impact of a digital Modified Checklist for Autism in Toddlers-Revised on likelihood and age of autism diagnosis and referral for developmental evaluation. Autism. 2020; 24(7):1629-38. PMID: 32466674; PMCID: PMC7541635.
67. de Nocker Y L, Toolan C K. Using Telehealth to Provide Interventions for Children with ASD: a Systematic Review. Rev J Autism Dev Disord. 2021; July 16 (Epub ahead of print): 1-31. PMID: 34306962; PMCID: PMC8282771.
68. Alfuraydan M, Croxall J, Hurt L, Kerr M, Brophy S. Use of telehealth for facilitating the diagnostic assessment of Autism Spectrum Disorder (ASD): A scoping review. PLOS One. 2020; 15(7):e0236415. PMID: 32702017; PMCID: PMC7377392.
69. Pi H J, Kallapiran K, Munivenkatappa S, Kandasamy P, Kirubakaran R, Russell P, Eapen V. Meta-Analysis of RCTs of Technology-Assisted Parent-Mediated Interventions for Children with ASD. J Autism Dev Disord. 2021; July (Epub ahead of print). PMID: 34318434.
70. Vismara L A, Young G S, Rogers S J. Telehealth for expanding the reach of early autism training to parents. Autism Res Treat. 2012; 2012:121878. PMID: 23227334; PMCID: PMC3512210.
71. Bangerter A, Manyakov N V, Lewin D, Boice M, Skalkin A, Jagannatha S, Chatterjee M, Dawson G, Goodwin M S, Hendren R, Leventhal B, Shic F, Ness S, Pandina G. Caregiver Daily Reporting of Symptoms in Autism Spectrum Disorder: Observational Study Using Web and Mobile Apps. JMIR Ment Health. 2019; 6(3):e11365. PMID: 30912762; PMCID: PMC6454343.
72. SPARK Consortium. SPARK: A US Cohort of 50,000 Families to Accelerate Autism Research. Neuron. 2018; 97(3):488-93. PMID: 29420931; PMCID: PMC7444276.
73. Levy D L, Sereno A B, Gooding D C, O'Driscoll G A. Eye tracking dysfunction in schizophrenia: characterization and pathophysiology. Curr Top Behav Neurosci. 2010; 4:311-47. PMID: 21312405; PMCID: PMC3212396.
74. Tao L, Wang Q, Liu D, Wang J, Zhu Z, Feng L. Eye tracking metrics to screen and assess cognitive impairment in patients with neurological disorders. Neurol Sci. 2020; 41(7):1697-704. PMID: 32125540.
75. Dawson G, Bernier R. A quarter century of progress on the early detection and treatment of autism spectrum disorder. Dev Psychopathol. 2013; 25(4 Pt 2):1455-72. PMID: 24342850.
76. Daniels A M, Halladay A K, Shih A, Elder L M, Dawson G. Approaches to enhancing the early detection of autism spectrum disorders: a systematic review of the literature. J Am Acad Child Adolesc Psychiatry. 2014; 53(2):141-52. PMID: 24472250.
77. Chang Z, Di Martino J M, Qiu Q, Espinosa S, Sapiro G. SalGaze: Personalizing gaze estimation using visual saliency. IEEE/CVF International Conference on Computer Vision Workshops. 2019:1169-78.
78. Sabatos-DeVito M, Murias M, Dawson G, Howell T, Yuan A, Marsan S, Bernier R A, Brandt C A, Chawarska K, Dzuira J D, Faja S, Jeste S S, Naples A, Nelson C A, Shic F, Sugar C A, Webb S J, McPartland J C. Methodological considerations in the use of Noldus EthoVision XT video tracking of children with autism in multi-site studies. Biol Psychol. 2019; 146:107712. PMID: 31163191; PMCID: PMC7334026.
79. Franz L, Dawson G. Implementing early intervention for autism spectrum disorder: a global perspective. Pediatric Medicine. 2019; 2. PMID: 31535090; PMCID: PMC6750262.
80. Ramseur K, 2nd, de Vries P J, Guler J, Shabalala N, Seris N, Franz L. Caregiver descriptions of joint activity routines with young children with autism spectrum disorder in South Africa. Pediatric Medicine. 2019; 2:6. PMID: 31032484; PMCID: PMC6481954.
81. Goldstein B A, Cerullo M, Krishnamoorthy V, Blitz J, Mureebe L, Webster W, Dunston F, Stirling A, Gagnon J, Scales C D, Jr. Development and Performance of a Clinical Decision Support Tool to Inform Resource Utilization for Elective Operations. JAMA Netw Open. 2020; 3(11):e2023547. PMID: 33136133; PMCID: PMC7607444.
82. Goldstein B A, Bedoya A D. Guiding Clinical Decisions Through Predictive Risk Rules. JAMA Netw Open. 2020; 3(8):e2013101. PMID: 32780120.
83. Hatch B, Iosif A M, Chuang A, de la Paz L, Ozonoff S, Miller M. Longitudinal Differences in Response to Name Among Infants Developing ASD and Risk for ADHD. J Autism Dev Disord. 2021; 51(3):827-36. PMID: 31974800; PMCID: PMC7375942.
84. Miller M, Iosif A M, Hill M, Young G S, Schwichtenberg A J, Ozonoff S. Response to Name in Infants Developing Autism Spectrum Disorder: A Prospective Study. J Pediatr. 2017; 183:141-6.e1. PMID: 28162768; PMCID: PMC5370582.
85. Nadig A S, Ozonoff S, Young G S, Rozga A, Sigman M, Rogers S J. A prospective study of response to name in infants at risk for autism. Arch Pediatr Adolesc Med. 2007; 161(4):378-83. PMID: 17404135.
86. Bangerter A, Chatterjee M, Manfredonia J, Manyakov N V, Ness S, Boice M A, Skalkin A, Goodwin M S, Dawson G, Hendren R, Leventhal B, Shic F, Pandina G. Automated recognition of spontaneous facial expression in individuals with autism spectrum disorder: parsing response variability. Mol Autism. 2020; 11(1):31. PMID: 32393350; PMCID: PMC7212683.
87. Trevisan D A, Hoskyn M, Birmingham E. Facial Expression Production in Autism: A Meta-Analysis. Autism Res. 2018; 11(12):1586-601. PMID: 30393953.
88. Diagnostic and Statistical Manual of Mental Disorders (5th Edition). Washington, DC: American Psychiatric Association; 2013.
89. Sapiro G. Computer vision tools for low-cost and noninvasive measurement of autism-related behaviors in infants. Autism Res Treat. 2014; 2014:935686. PMID: 25045536; PMCID: PMC4090521.
90. Iverson J M, Shic F, Wall C A, Chawarska K, Curtin S, Estes A, Gardner J M, Hutman T, Landa R J, Levin A R, Libertus K, Messinger D S, Nelson C A, Ozonoff S, Sacrey L R, Sheperd K, Stone W L, Tager-Flusberg H B, Wolff J J, Yirmiya N, Young G S. Early motor abilities in infants at heightened versus low risk for ASD: A Baby Siblings Research Consortium (BSRC) Study. J Abnorm Psychol. 2019; 128(1):69-80. PMID: 30628809; PMCID: PMC6338079.
91. Choi B, Leech K A, Tager-Flusberg H, Nelson C A. Development of fine motor skills is associated with expressive language outcomes in infants at high and low risk for autism spectrum disorder. J Neurodev Disord. 2018; 10(1):14. PMID: 29649977; PMCID: PMC5898056.
92. Ghanouni P, Memari A H, Gharibzadeh S, Eghlidi J, Moshayedi P. Effect of Social Stimuli on Postural Responses in Individuals with Autism Spectrum Disorder. J Autism Dev Disord. 2017; 47(5):1305-13. PMID: 28236101.
93. Minshew N J, Sung K, Jones B L, Furman J M. Underdevelopment of the postural control system in autism. Neurology. 2004; 63(11):2056-61. PMID: 15596750.
94. Gouleme N, Scheid I, Peyre H, Seassau M, Maruani A, Clarke J, Delorme R, Bucci M P. Postural Control and Emotion in Children with Autism Spectrum Disorders. Transl Neurosci. 2017; 8:158-66. PMID: 29177103; PMCID: PMC5700205.
95. Cham R, Iverson J M, Bailes A H, Jennings J R, Eack S M, Redfern M S. Attention and sensory integration for postural control in young adults with autism spectrum disorders. Exp Brain Res. 2021; 239(5):1417-26. PMID: 33675379.
96. Shumway-Cook A, Woollacott M. Attentional demands and postural control: the effect of sensory context. J Gerontol A Biol Sci Med Sci. 2000; 55(1):M10-6. PMID: 10719767.
97. Lord C, Rutter M, DiLavore P, Risi S, Gotham K, Bishop S. Autism Diagnostic Observation Schedule, Second Edition (ADOS-2). Torrance, CA: Western Psychological Services; 2012.
98. Adamson L B, Bakeman R, Suma K. The Joint Engagement Rating Inventory. Department of Psychology, Georgia State University. 2020.
99. Adamson L B, Bakeman R, Suma K, Robins D L. An Expanded View of Joint Attention: Skill, Engagement, and Language in Typical Development and Autism. Child Dev. 2019; 90(1):e1-e18. PMID: 28991358; PMCID: PMC5891390.
100. Kenny D A, Kashy D A, Cook W L. Dyadic Data Analysis. Guilford Press. 2006.
101. Fuchs P, Nussbeck F W, Meuwly N, Bodenmann G. Analyzing Dyadic Sequence Data—Research Questions and Implied Statistical Models. Front Psychol. 2017; 8:429. PMID: 28443037; PMCID: PMC5387096.
102. Van de Pol F, Langeheine R. Mixed Markov Latent Class Models. Sociol Methodol. 1990; 20:213-47.
103. Sparrow S, Cicchetti D, Saulnier C A. Vineland Adaptive Behavior Scale—Third Edition. Pearson. 2016. health care coverage of children in Durham County, NC. Annual Meeting of the Population Association of America. May 2021; St. Louis, Missouri.
105. Coleman K J, Lutsky M A, Yau V, Qian Y, Pomichowski M E, Crawford P M, Lynch F L, Madden J M, Owen-Smith A, Pearson J A, Pearson K A, Rusinak D, Quinn V P, Croen L A. Validation of Autism Spectrum Disorder Diagnoses in Large Healthcare Systems with Electronic Medical Records. J Autism Dev Disord. 2015; 45(7):1989-96. PMID: 25641003; PMCID: PMC4474741.
106. Perrin E C, Sheldrick C, Visco Z, Mattern K. The Survey of Well-being of Young Children (SWYC). Tufts Medical Center. 2016.
107. Sheldrick R C, Marakovitz S, Garfinkel D, Carter A S, Perrin E C. Comparative Accuracy of Developmental Screening Questionnaires. JAMA Pediatr. 2020; 174(4):366-74. PMID: 32065615; PMCID: PMC7042946.
108. Sheldrick R C, Perrin E C. Evidence-based milestones for surveillance of cognitive, language, and motor development. Acad Pediatr. 2013; 13(6):577-86. PMID: 24238685; PMCID: PMC3993976.
109. Lipkin P H, Macias M M. Promoting Optimal Development: Identifying Infants and Young Children With Developmental Disorders Through Developmental Surveillance and Screening. Pediatrics. 2020; 145(1). PMID: 31843861.
110. Achenbach T M, Rescorla L A. Manual for the Achenbach System of Empirically Based Assessment (ASEBA) Preschool Forms & Profiles. Burlington, VT: University of Vermont Research Center for Children, Youth, & Families; 2000.
111. Constantino J. Social Responsiveness Scale, 2nd Edition (SRS-2). Torrance, CA: Western Psychological Services; 2012.
112. Constantino J N, Davis S A, Todd R D, Schindler M K, Gross M M, Brophy S L, Metzger L M, Shoushtari C S, Splinter R, Reich W. Validation of a brief quantitative measure of autistic traits: comparison of the social responsiveness scale with the autism diagnostic interview-revised. J Autism Dev Disord. 2003; 33(4):427-33. PMID: 12959421.
113. Rutter M, Le Couteur A, Lord C. Autism Diagnostic Interview-Revised (ADI-R). Torrance, CA: Western Psychological Services; 2003.
114. Wagner L, Stone C, Wade J, Corona L, Hine J, Nicholson A, Swanson A, Vehorn A, Weitlauf A, Warren Z. TELE-ASD-PEDS: A Telemedicine-based ASD Evaluation Tool for Toddlers and Young Children. Vanderbilt University Medical Center. 2021.
115. Wagner L, Corona L L, Weitlauf A S, Marsh K L, Berman A F, Broderick N A, Francis S, Hine J, Nicholson A, Stone C, Warren Z. Use of the TELE-ASD-PEDS for Autism Evaluations in Response to COVID-19: Preliminary Outcomes and Clinician Acceptability. J Autism Dev Disord. 2021; 51(9):3063-72. PMID: 33125622; PMCID: PMC7596626.
116. Wagner L, Weitlauf A S, Hine J, Corona L L, Berman A F, Nicholson A, Allen W, Black M, Warren Z. Transitioning to Telemedicine During COVID-19: Impact on Perceptions and Use of Telemedicine Procedures for the Diagnosis of Autism in Toddlers. J Autism Dev Disord. 2021; June 4 (Epub ahead of print): 1-11. PMID: 34085153; PMCID: PMC8175230.
117. Berger N I, Wainer A L, Kuhn J, Bearss K, Attar S, Carter A S, Ibanez L V, Ingersoll B R, Neiderman H, Scott S, Stone W L. Characterizing Available Tools for Synchronous Virtual Assessment of Toddlers with Suspected Autism Spectrum Disorder: A Brief Report. J Autism Dev Disord. 2021:1-12. PMID: 33606157; PMCID: PMC7893832.
118. Juárez A P, Weitlauf A S, Nicholson A, Pasternak A, Broderick N, Hine J, Stainbrook J A, Warren Z. Early Identification of ASD Through Telemedicine: Potential Value for Underserved Populations. J Autism Dev Disord. 2018; 48(8):2601-10. PMID: 29527626; PMCID: PMC6041148.
119. Khowaja M K, Hazzard A P, Robins D L. Sociodemographic Barriers to Early Detection of Autism: Screening and Evaluation Using the M-CHAT, M-CHAT-R, and Follow-Up. J Autism Dev Disord. 2015; 45(6):1797-808. PMID: 25488122; PMCID: PMC4442731.
120. Lord C, Dow D, Holbrook A, Kim S. Brief Observation of Symptoms of Autism (BOSA). UCLA Semel Institute Center for Autism Research & Treatment. 2020.
121. Voress J K, Maddox T. Developmental Assessment of Young Children-Second Edition (DAYC-2). PRO-ED. 2013.
122. Andersson L. Use of the Communication Development Subtest of the DAYC as a Teacher-Administered Language-Screening Instrument. Communications Disorders Quarterly. 2006; 27(4):206-12.
123. King D E. Dlib-ml: A machine learning toolkit. J Machine Learning Research. 2009; 10:1755-8.
124. De la Torre F, Chu W, Xiong X, Vincente F, Ding X, Cohn J. IntraFace. IEEE International Conference on Automatic Face and Gesture Recognition and Workshops. 2015; 1. PMID: 27346987; PMCID: PMC4918819.
125. Iranmanesh S M, Dabouei A, Soleymani S, Kazemi H, Nasrabadi N M. Robust facial landmark detection via aggregation on geometrically manipulated faces. Proceedings of the IEEE/CVF Winter Conference on Applications of Computer Vision. 2020:330-40.
126. Yan Y, Duffner S, Phutane P, Berthelier A, Naturel X, Blanc C, Chateau T. Fine-grained facial landmark detection exploiting intermediate feature representations. Computer Vision and Image Understanding. 2020; 200.
127. Lugaresi C, Tang J, Nash H, McClanahan C, Uboweja E, Hays M, Zhang F, Chang C-L, Yong M G, Lee J, Chang W-T, Hua W, Georg M, Grundmann M. MediaPipe: A framework for building perception pipelines. arXiv: 190608172. 2019.
128. Baltrusaitis T, Robinson P, Morency L P. Openface: an open source facial behavior analysis toolkit. IEEE Winter Conference on Applications of Computer Vision (WACV). 2016:1-10.
129. Baltrusaitis T, Zadeh A, Lim Y C, Morency L P. Openface 2.0: Facial behavior analysis toolkit. IEEE International Conference on Automatic Face & Gesture Recognition. 2018:59-66.
130. Tenenbaum E J, Carpenter K L H, Sabatos-DeVito M, Hashemi J, Vermeer S, Sapiro G, Dawson G. A Six-Minute Measure of Vocalizations in Toddlers with Autism Spectrum Disorder. Autism Res. 2020; 13(8):1373-82. PMID: 32212384; PMCID: PMC7881362.
131. Cao Z, Hidalgo G, Simon T, Wei S E, Sheikh Y. OpenPose: Realtime Multi-Person 2D Pose Estimation Using Part Affinity Fields. IEEE Transactions on Pattern Analysis and Machine Intelligence. 2021; 43(1):172-86.
132. Güler R A, Neverova N, Kokkinos I. DensePose: Dense Human Pose Estimation in the Wild. Proceedings of the IEEE Computer Society Conference on Computer Vision and Pattern Recognition. 2018:7297-306.
133. Moon G, Chang J Y, Lee K M. Camera distance-aware top-down approach for 3D multi-person pose estimation from a single RGB image. Proceedings of the IEEE International Conference on Computer Vision. 2019:10132-41.
134. Maguire M. Methods to support human-centred design. Int J Human-Computer Studies. 2001; 55(4):587-634.
135. Palinkas L A, Horwitz S M, Green C A, Wisdom J P, Duan N, Hoagwood K. Purposeful Sampling for Qualitative Data Collection and Analysis in Mixed Method Implementation Research. Adm Policy Ment Health. 2015; 42(5):533-44. PMID: 24193818; PMCID: PMC4012002.
136. Hsieh H F, Shannon S E. Three approaches to qualitative content analysis. Qual Health Res. 2005; 15(9):1277-88. PMID: 16204405.
137. Lyon A R, Munson S A, Renn B N, Atkins D C, Pullmann M D, Friedman E, Arean P A. Use of Human-Centered Design to Improve Implementation of Evidence-Based Psychotherapies in Low-Resource Communities: Protocol for Studies Applying a Framework to Assess Usability. JMIR Res Protoc. 2019; 8(10):e14990. PMID: 31599736; PMCID: PMC6819011.
138. Lewis J R, Sauro J. The Factor Structure of the System Usability Scale. Proceedings of First International Conference on Human Centered Design. 2009; 10 (LNCS 5619):94-103.
139. Sun Y, Wong A K, Kamel M S. Classification of imbalanced data: A review. International Journal of Pattern Recognition and Artificial Intelligence. 2009; 23(4):687-719.
140. Barandela R, Sánchez J S, Garc a V, Rangel E. Strategies for learning in class imbalance problems. Pattern Recognition. 2003; 36(3):849-51.
141. Ali H, Mohd Salleh M N, Saedudin R, Hussain K, Mushtaq M F. Imbalance class problems in data mining: a review. Indonesian Journal of Electrical Engineering and Computer Science. 2019; 14(3):1552.
142. Japkowicz N, Stephen S. The class imbalance problem: A systematic study. Intelligent Data Analysis. 2002; 6(5):429. PMID: 8769737.
143. Batuwita R, Palade V. A New Performance Measure for Class Imbalance Learning: Application to Bioinformatics Problems. Proceedings of the 2009 International Conference on Machine Learning and Applications. 2009:545-50.
144. Lemaître G, Nogueira F, Aridas C K. Imbalanced-learn: A python toolbox to tackle the curse of imbalanced datasets in machine learning. Journal of Machine Learning Research. 2017; 18(1):559-63.
145. Pedregosa F, Varoquaux G, Gramfort A, Michel V, Thirion B, Grisel O, Blondel M, Prettenhofer P, Weiss R, Dubourg V, Vanderplas J, Passos A, Cournapeau D, Brucher M, Perrot M, Duchesnay E. Scikit-learn: Machine Learning in Python. Journal of Machine Learning Research. 2011; 12:2825-30.
146. Di Martino M, Hernández G, Fiori M, Fernandez A. A new framework for optimal classifier design. Pattern Recognition. 2013; 46(8):2249-55.
147. Fiori M, Di Martino M, Fernandez A. An optimal multiclass classifier design. Proceedings of 23^{rd} International Conference on Pattern Recognition (ICPR). 2016:480-5.
148. Hong X, Chen S, Harris C J. A Kernel-Based Two-Class Classifier for Imbalanced Data Sets. IEEE Transactions on Neural Networks. 2007; 18(1):28-41.
149. Kay S M. Fundamentals of Statistical Signal Processing. Upper Saddle River, NJ: Prentice Hall PTR; 1993.
150. Wasserman L. All of Statistics: A Concise Course in Statistical Inference. New York: Springer Science+Business Media; 2004.
151. Revelle W. Procedures for Psychological, Psychometric, and Personality Research (psych), Version 2.1.3. 2021.
152. Koo T K, Li M Y. A Guideline of Selecting and Reporting Intraclass Correlation Coefficients for Reliability Research. J Chiropr Med. 2016; 15(2):155-63.
153. Bates D, Maechler M, Bolker B, Walker S, Christensen R H B, Singmann H, Dai B, Scheipl F, Grothendieck G. Linear Mixed-Effects Models Using ‘Eigen’ and S4 (Ime4). 2021.
154. Pinheiro J, Bates D, DebRoy S, Sarkar D, Heisterkamp S, Van Willigen B, Ranke J. Linear and Nonlinear Mixed Effects Models (nIme), Version 3.1-152. 2021.
155. Fox J. TEACHER'S CORNER: Structural Equation Modeling With the sem Package in R. Structural Equation Modeling: A Multidisciplinary Journal. 2006; 13(3):465-86.
156. Rosseel Y. Lavaan: An R package for structural equation modeling and more. Version 0.5-12 (BETA). Journal of Statistical Software. 2012; 48(2):1-36.
157. Schoonenboom J, Johnson R B. How to Construct a Mixed Methods Research Design. Kolner Z Soz Sozpsychol. 2017; 69(Suppl 2):107-31. PMID: 28989188; PMCID: PMC5602001.
158. Faul F, Erdfelder E, Lang A G, Buchner A. G*Power 3: a flexible statistical power analysis program for the social, behavioral, and biomedical sciences. Behav Res Methods. 2007; 39(2):175-91. PMID: 17695343.
159. Spybrook J, Raudenbush S W, Liu X F, Congdon R, Martínez A. Optimal design for longitudinal and multilevel research: Documentation for the “Optimal Design” software. Survey Research Center of the Institute of Social Research at University of Michigan. 2006.
160. Bujang M A, Baharum N. A simplified guide to determination of sample size requirements for estimating the value of intraclass correlation coefficient: a review. Archives of Orofacial Science. 2017; 12(1):1-11.
161. Simel D L, Samsa G P, Matchar D B. Likelihood ratios with confidence: Sample size estimation for diagnostic test studies. J Clin Epidemiol. 1991; 44(8):763-70. PMID: 1941027.
162. Stark M, Zapf A. Sample size calculation and re-estimation based on the prevalence in a single-arm confirmatory diagnostic accuracy study. Stat Methods Med Res. 2020; 29(10):2958-71.
163. Cicchetti D V. The precision of reliability and validity estimates re-visited: distinguishing between clinical and statistical significance of sample size requirements. J Clin Exp Neuropsychol. 2001; 23(5):695-700. PMID: 11778646.
164. Hoyle R H, Gottfredson N C. Sample Size Considerations in Prevention Research Applications of Multilevel Modeling and Structural Equation Modeling. Prevention Science. 2015; 16(7):987-96. PMID: 24752569; PMCID: PMC4207737.
165. Kyriazos T A. Applied Psychometrics: Sample Size and Sample Power Considerations in Factor Analysis (EFA, CFA) and SEM in General. Psychology. 2018; 9(8):2207-30.

While at least one example embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the example embodiment(s). In addition, in this disclosure, the terms “comprise” or “comprising” do not exclude other elements or steps, the terms “a”, “an” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

What is claimed is:

1. A method of detecting risk of neurodevelopmental disorders, the method comprising:
collecting patient information;
deriving a risk level from the patient information; and
providing next-step guidance corresponding to the risk level;
wherein collecting the patient information comprises extracting information from one or more outputs selected from the group consisting of an electronically-administered screening survey; electronic health records information; direct observation of the patient via an application delivered on a computer, tablet, or smartphone; a phenotypic algorithm; predictive markers; genetic testing; and any combination thereof.

2. The method of claim 1, wherein the predictive markers comprise patterns of early medical conditions.

3. The method of claim 2, wherein a machine learning algorithm is used to identify the predictive markers.

4. The method of claim 3, wherein the machine learning algorithm comprises a transformer-based neural network pre-trained on biomedical text.

5. The method of claim 1, wherein extracting information from direct observation of the patient comprises:
observing the patient via the application using computer vision analysis; and
transmitting the patient information from the computer, tablet, or smartphone to a central healthcare data system.

6. The method of claim 1, wherein extracting information from a phenotypic algorithm comprises collecting data from computer vision analysis of activities selected from the group consisting of response to name, facial expression and dynamics, postural control and fine motor skills, social attention, and any combination thereof.

7. The method of claim 1, wherein deriving the risk level comprises:
deriving individual risk assessments from each of the one or more outputs; and
aggregating the individual risk assessments to determine the risk level.

8. The method of claim 7, wherein aggregating the individual risk assessments comprises assigning weights to the individual risk assessments based on outputs from a machine learning algorithm.

9. The method of claim 1, wherein providing next-step guidance comprises interfacing with a central healthcare data system to deliver recommendations for referrals and/or treatment.

10. The method of claim 1, wherein providing next-step guidance comprises integrating strategies and/or monitoring to the caregiver and/or patient within a routine schedule of care.

11. A system for detecting and/or monitoring risk of neurodevelopmental disorders, comprising a computing system configured to perform the method of claim 1.
